(19) United States
(12) Patent Application Publication
(10) Pub. No.: US 2024/0402113 A1
(43) Pub. Date: Dec. 5, 2024

(54) THERMO-ELECTRICAL WIRELESS SENSOR FOR HIGH-FREQUENCY ELECTRICAL IMPEDANCE WITH SOLAR RECHARGE

(71) Applicant: Sensytec, Inc., Houston, TX (US)

(72) Inventors: Sai Anudeep Reddy Maddi, Coatesville, PA (US); Derman Smith Santander Amador, Houston, TX (US); Ody De La Paz Guerra, Houston, TX (US); Roberto D. Morales, Houston, TX (US)

(21) Appl. No.: 18/794,309

(22) Filed: Aug. 5, 2024

Related U.S. Application Data
(63) Continuation-in-part of application No. 18/171,767, filed on Feb. 21, 2023, now Pat. No. 12,055,505.
Application No. 18/171,767 is a continuation of application No. 17/380,115, filed on Jul. 20, 2021, now Pat. No. 11,614,420.
(60) Provisional application No. 63/054,283, filed on Jul. 21, 2020.

(51) Int. Cl. G01N 27/04; G01N 33/38

(52) U.S. Cl. CPC G01N 27/046; G01N 33/383

(57) ABSTRACT

The present disclosure provides a sensor system comprising a sensor node and a detachably attachable sensor probe. The sensor node includes a sensor enclosure, a solar panel, at least one probe connection port, and processing circuitry. The sensor probe includes a probe enclosure, a thermocouple for temperature measurement, at least two conductive contact points for electrical measurements, and a cable connecting to the sensor node. The processing circuitry receives temperature data from the thermocouple and electrical measurement data from the conductive contact points, processes the received data, and wirelessly transmits the processed data to an external device. The solar panel powers the sensor node for autonomous operation. The system enables long-term monitoring of materials such as concrete, soil, wood, and polymers, providing real-time data on temperature, electrical properties, and material characteristics.

### CROSS-REFERENCE TO RELATED APPLICATIONS

This application is a continuation-in-part of U.S. patent application Ser. No. 18/171,767 filed Feb. 21, 2023, now issued as U.S. Pat. No. 12,055,505, which is a continuation of U.S. patent application Ser. No. 17/380,115 filed Jul. 20, 2021, now issued as U.S. Pat. No. 11,614,420, which claims the benefit of and priority to U.S. Provisional Patent Application No. 63/054,283 filed Jul. 21, 2020, the contents of each of which being incorporated by reference in their entireties herein.

### TECHNICAL FIELD

The present disclosure relates generally to sensor systems and, more particularly, to a thermo-electrical wireless sensor system utilizing high-frequency electrical impedance measurements with solar recharge capability for long-term monitoring of various materials and environments. In some aspects, the disclosure may pertain to real-time monitoring of concrete, soil, and other mediums using embedded sensors that measure temperature and electrical properties. The system may find applications in fields such as construction, civil engineering, geotechnical engineering, environmental monitoring, and structural health assessment.

### BACKGROUND

In many fields, there is a need for continuous monitoring of parameters over long durations. Traditional monitoring systems often provide data intermittently or require manual intervention for data collection and analysis. Real-time long-term monitoring systems overcome these limitations by providing continuous data collection and immediate analysis of the collected data.

Wireless sensor networks have gained prominence in various industries for their ability to remotely monitor environmental and structural parameters. Long Range technology provides an efficient means of wireless communication over long distances, suitable for applications requiring low power consumption and extended battery life. Monitoring temperature and electrical resistivity, especially using high-frequency electrical impedance measurements, is crucial in fields such as agriculture, construction, environmental monitoring, and industrial process control.

The integration of wireless communication technology, high-frequency electrical impedance measurement, and solar recharge capability opens up new possibilities for autonomous, long-term monitoring systems. Such systems can provide valuable insights into material properties, structural health, and environmental conditions without the need for frequent maintenance or battery replacement. However, challenges remain in developing robust, energy-efficient sensors that can operate reliably in diverse environments while providing accurate and timely data for analysis and decision-making.

### BRIEF SUMMARY

According to an aspect of the present disclosure, a sensor system is provided. The sensor system includes a sensor node comprising a sensor enclosure, a solar panel positioned on a surface of the sensor enclosure, at least one probe connection port, and processing circuitry housed within the sensor enclosure. The sensor system also includes a sensor probe detachably attachable to the sensor node. The sensor probe comprises a probe enclosure, a thermocouple positioned within the probe enclosure for temperature measurement, at least two conductive contact points positioned within the probe enclosure for electrical measurements, and a cable connecting the probe enclosure to the sensor node via the at least one probe connection port. The processing circuitry is configured to receive temperature data from the thermocouple, receive electrical measurement data from the at least two conductive contact points, process the received temperature data and electrical measurement data, and wirelessly transmit the processed data to an external device.

According to other aspects of the present disclosure, the sensor system may include one or more of the following features. The processing circuitry may be further configured to perform high-frequency electrical impedance measurements using the at least two conductive contact points and calculate electrical resistivity of a surrounding medium based on the high-frequency electrical impedance measurements. The solar panel may be configured to provide power to the sensor node for autonomous operation. The sensor node may further comprise a rechargeable battery charged by the solar panel. The processing circuitry may be further configured to implement machine learning algorithms to analyze the processed data and predict future conditions of a monitored medium. The at least two conductive contact points may be gold-coated metal contact points (or gold-coated metal pins). The sensor node may comprise multiple probe connection ports for connecting multiple sensor probes. The processing circuitry may be further configured to determine at least one of water-to-cement ratio, compressive strength, setting time, or transport properties of concrete based on the processed data. The sensor probe may be configured to be embedded in a material selected from the group consisting of concrete, soil, wood, and polymers. The sensor system may further comprise a gateway device configured to receive the wirelessly transmitted processed data from the sensor node and relay the processed data to a remote server for further analysis and visualization.

According to another aspect of the present disclosure, a method for monitoring a material using a sensor system is provided. The method includes providing a sensor node comprising a sensor enclosure, a solar panel positioned on a surface of the sensor enclosure, at least one probe connection port, and processing circuitry housed within the sensor enclosure. The method also includes providing a sensor probe detachably attachable to the sensor node, the sensor probe comprising a probe enclosure, a thermocouple positioned within the probe enclosure for temperature measurement, at least two conductive contact points positioned within the probe enclosure for electrical measurements, and a cable connecting the probe enclosure to the sensor node via the at least one probe connection port. The method further includes receiving, by the processing circuitry, temperature data from the thermocouple, receiving, by the processing circuitry, electrical measurement data from the at least two conductive contact points, processing, by the processing circuitry, the received temperature data and electrical measurement data, and wirelessly transmitting, by the processing circuitry, the processed data to an external device.

According to other aspects of the present disclosure, the method may include one or more of the following features. The method may further comprise performing, by the processing circuitry, high-frequency electrical impedance measurements using the at least two conductive contact points, and calculating, by the processing circuitry, electrical resistivity of a surrounding medium based on the high-frequency electrical impedance measurements. The method may further comprise powering the sensor node using the solar panel for autonomous operation. The method may further comprise charging a rechargeable battery of the sensor node using the solar panel. The method may further comprise implementing, by the processing circuitry, machine learning algorithms to analyze the processed data and predict future conditions of a monitored medium. The at least two conductive contact points may be gold-coated metal contact points. The method may further comprise connecting multiple sensor probes to the sensor node via multiple probe connection ports. The method may further comprise determining, by the processing circuitry, at least one of water-to-cement ratio, compressive strength, setting time, or transport properties of concrete based on the processed data. The method may further comprise embedding the sensor probe in a material selected from the group consisting of concrete, soil, wood, and polymers. The method may further comprise receiving, by a gateway device, the wirelessly transmitted processed data from the sensor node, and relaying, by the gateway device, the processed data to a remote server for further analysis and visualization.

According to another aspect of the present disclosure, a sensor system is provided. The sensor system includes a sensor node comprising a sensor enclosure, a solar panel positioned on a surface of the sensor enclosure, at least one probe connection port, and processing circuitry housed within the sensor enclosure. The sensor system also includes a sensor probe detachably attachable to the sensor node. The sensor probe comprises a probe enclosure, a thermocouple positioned within the probe enclosure for temperature measurement, at least two conductive contact points positioned within the probe enclosure for electrical measurements, and a cable connecting the probe enclosure to the sensor node via the at least one probe connection port. The processing circuitry is configured to receive temperature data from the thermocouple, receive electrical measurement data from the at least two conductive contact points, process the received temperature data and electrical measurement data using a machine learning model, generate predictions related to a monitored medium based on the processed data, and wirelessly transmit the processed data and predictions to an external device.

According to other aspects of the present disclosure, the sensor system may include one or more of the following features. The machine learning model may be trained to predict at least one of: future temperature changes, future electrical property changes, material strength development, or potential structural issues in the monitored medium. The processing circuitry may be further configured to adaptively calibrate the sensor probe based on historical data and current measurements using the machine learning model. The machine learning model may be configured to detect anomalies in the received temperature data and electrical measurement data that indicate potential sensor failures or degradation. The processing circuitry may be further configured to update the machine learning model based on new data received from the sensor probe. The machine learning model may be configured to estimate at least one of: water-to-cement ratio, compressive strength, setting time, or transport properties of concrete based on the processed data. The machine learning model may be configured to predict the onset of structural issues in the monitored medium based on analyzed trends in the processed data. The processing circuitry may be further configured to implement transfer learning techniques to adapt the machine learning model for different types of monitored media. The machine learning model may comprise at least one of: a neural network, a decision tree, a random forest, or a support vector machine. The processing circuitry may be further configured to perform feature extraction on the received temperature data and electrical measurement data before inputting the data into the machine learning model.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 is a perspective view of a sensor device for real-time concrete monitoring according to various embodiments of the present disclosure.

FIG. 2 is another perspective view of the sensor device according to various embodiments of the present disclosure.

FIG. 3 is a waterproof electrical connector for precast applications according to various embodiments of the present disclosure.

FIG. 4A is a copper electrode according to various embodiments of the present disclosure.

FIG. 4B is a plastic mixing arm according to various embodiments of the present disclosure.

FIG. 4C is a porous plastic filter according to various embodiments of the present disclosure.

FIG. 5 is an interior layout of a printed circuit board (PCB) and a corresponding component list and description table according to various embodiments of the present disclosure.

FIG. 6 is an example of an installation process for installing a sensor in concrete formwork according to various embodiments of the present disclosure.

FIG. 7 is an example of pouring concrete after placement of a sensor in concrete formwork according to various embodiments of the present disclosure.

FIG. 8 is an example of communicating wirelessly with a sensor according to various embodiments of the present disclosure.

FIG. 9 is a temperature plot showing temperature of concrete versus time according to various embodiments of the present disclosure.

FIG. 10 is a maturity plot showing maturity from temperature versus time according to various embodiments of the present disclosure.

FIG. 11 is a compressive strength plot showing compressive strength of concrete versus time according to various embodiments of the present disclosure.

FIG. 12 is an electrical resistivity plot showing electrical resistivity versus time according to various embodiments of the present disclosure.

FIG. 13 is a compressive strength using electrical resistivity plot showing compressive strength of concrete versus time according to various embodiments of the present disclosure.

FIG. 14 is an equivalent circuit diagram illustrating a total impedance of a sensor device embedded in concrete or other material according to various embodiments of the present disclosure.

FIG. 15 is a plot of impedance versus frequency of a circuit according to various embodiments of the present disclosure.

FIG. 16 is a schematic diagram that represents two independent testing parameters that portray a substantially linear relationship between electrical resistance and electrical resistivity.

FIG. 17 is a plot illustrating a linear relationship between electrical resistance and electrical resistivity according to various embodiments of the present disclosure.

FIG. 18 is a plot of electrical resistance versus electrical resistivity for concrete according to various embodiments of the present disclosure.

FIG. 19 is a plot of a K-value for concrete versus time according to various embodiments of the present disclosure.

FIG. 20 is a plot showing a resistivity versus time curve according to various embodiments of the present disclosure.

FIG. 21 is a plot showing a resistivity time factor according to various embodiments of the present disclosure.

FIG. 22 is a table showing a cumulative resistivity time factor versus compressive strength according to various embodiments of the present disclosure.

FIG. 23 is an isometric view of a sensor for real-time monitoring of concrete, soil, and other desirable mediums according to various embodiments of the present disclosure.

FIG. 24 is a schematic diagram of a sensor for real-time monitoring according to various embodiments of the present disclosure.

FIG. 25 is an orthogonal view of a sensor probe component for a sensor according to various embodiments of the present disclosure.

FIG. 26 is an isometric view of a sensor node for a thermo-piezoresistive embedded wireless sensor system according to various embodiments of the present disclosure.

FIG. 27 is another view of the sensor node showing its internal layout according to various embodiments of the present disclosure.

FIG. 28 is an isometric view of a sensor node for a sensor system according to various embodiments of the present disclosure.

FIG. 29 shows the sensor node in an outdoor environment according to various embodiments of the present disclosure.

FIG. 30 illustrates two perspective views of a gateway for use with a sensor and a solar panel that supplies power to the gateway according to various embodiments of the present disclosure.

FIG. 31 illustrates an installation of a thermoelectrical sensor system according to various embodiments of the present disclosure.

FIG. 32 illustrates a system diagram of a thermo-piezoresistive embedded wireless sensor network for real-time monitoring of concrete, soil, and other mediums according to various embodiments of the present disclosure.

FIG. 33 is a graph showing data from a thermo-piezoresistive embedded wireless sensor for real-time concrete monitoring according to various embodiments of the present disclosure.

### DETAILED DESCRIPTION

The present disclosure relates to a thermo-piezoresistive embedded wireless sensor with real-time concrete monitoring. Concrete material testing methods currently require concrete samples or “cylinders” to be collected from job sites for each of the concrete mix-designs used in a project. The samples are then carefully transported to a Materials Testing Lab (MTL) for testing. Typically, the MTL lets each concrete cylinder “specimen” cure for about three, seven, fourteen, or twenty-eight days before performing testing. Sequentially, after each concrete specimen is fully cured, the MTL performs a compressive strength test which includes performing various measurements by breaking each concrete cylinder in a compression-testing machine to determine at what pound-force per square inch (PSI) each concrete cylinder breaks. This assumes that the sample has not be broken during collection and/or transport.

The test results are sent to construction owners to inform the owners of the PSI of each concrete specimen, which is correlated to the PSI for concrete poured in the field. However, these testing methods have been around for decades, and they do not provide the necessary information in real-time for construction owners, general contractors, suppliers, regulators, architects, and civil engineers to make quick and more informed decisions about the performance and quality of concrete. Currently, the seven days lead time that MTL takes before sending results back to the end user is a hurdle to the construction industry where time is money.

Establishing rapid real-time testing of concrete quality and strength is the current aspect of interest. The use of thermocouples and sensors for temperature measurement in concrete to predict the strength are also accepted in the building standards for construction. Nonetheless, temperature measurements have proved to be accurate in controlled environment settings, but there are several limitations in applying the technology to the field. One limitation is the temperature being influenced by the outside environmental elements, such as hot and cold weather. Another limitation is that temperature measurements are affected by the size and thickness of the structure being built, and the location where a sensor is placed may affect readings due to the temperature of concrete varying from place to place. Notably, temperature sensors cannot differentiate between void space and the presence of concrete at the measurement location.

Furthermore, temperature measurements are not used solely for early concrete strength predictions due to temperature not being a true material property of concrete. On the other hand, electrical resistivity of concrete is a material property and provides accurate correlation to hydration reaction of concrete. This parameter can be used to predict a much more accurate strength predictions in fresh concrete. Estimation of strength and quality of concrete in real time using alternating current electrical resistivity measurements are described herein may provide contractors, owners, and others with highly accurate results, thereby reducing the dependence on break tests that are currently done, which cause longer construction waiting time and are expensive.

More details and features of the embodiments described herein will be described below. Specifically, the embodiments described herein address limitations within the current testing methods for concrete, cement, treated soils, solutions and polymers, more particularly electrical methods, and systems. Further, the embodiments described herein may utilize electrical impedance measurements to monitor the chemical reaction in concrete, such as fresh concrete, to determine the strength and quality thereof in real-time. The alternating current electrical impedance measurement may be performed at high-frequencies and may be used to calculate electrical resistivity based on a correction to geometric factor K—temperature adjustment. The embodiments described herein may provide electrical conductivity of pore solution inside cementitious materials in real time.

Other aspects and features of the present invention will become apparent to those ordinarily skilled in the art upon review of the following description of specific embodiments of the invention in conjunction with the accompanying figures.

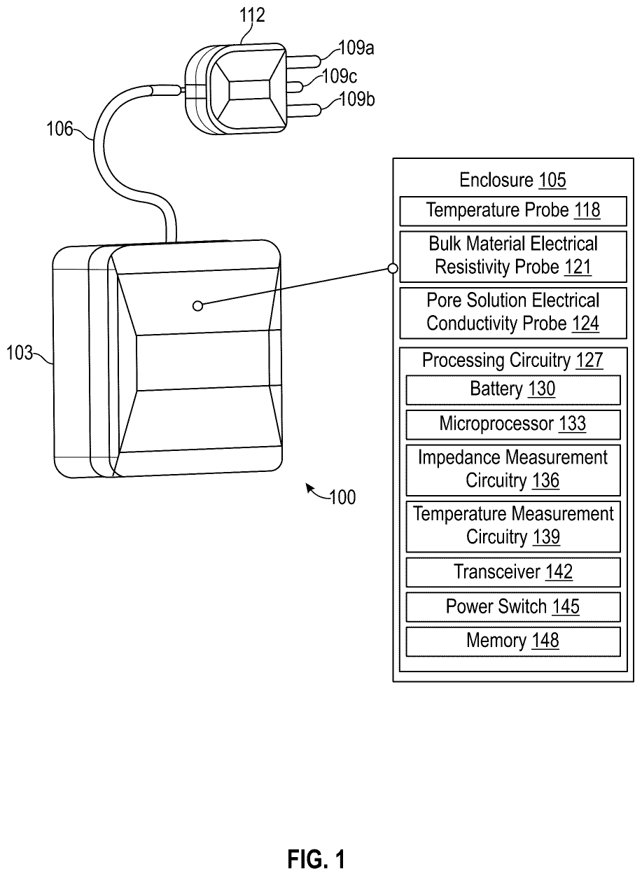

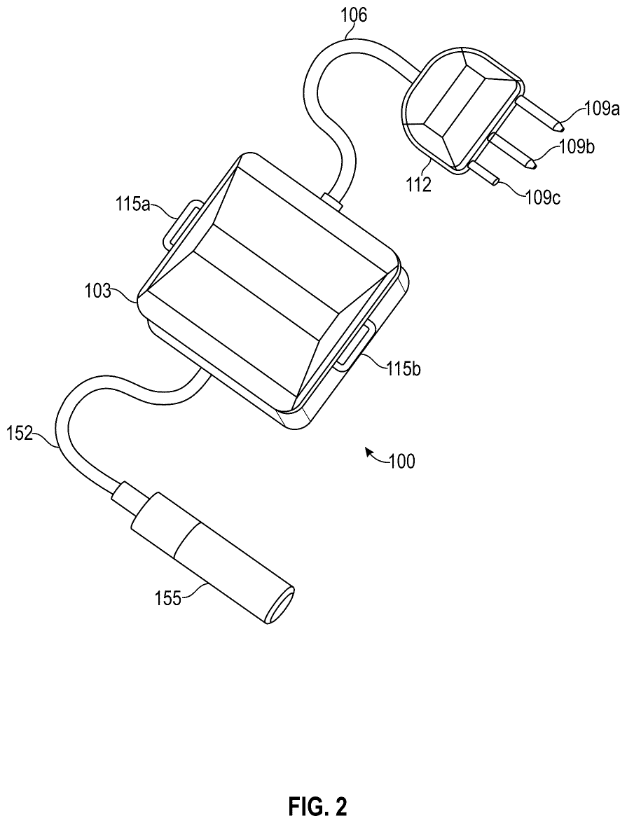

Referring now to FIGS. 1 and 2, non-limiting examples of a sensor device 100 are shown in accordance with various embodiments. The sensor device 100 is configured to be embedded in concrete, dirt, or other construction material while still being able to capture measurements of the construction material or properties thereof and communicate the measurements to a computing device outside of the construction material.

In some embodiments, the sensor device 100 includes an enclosure 103, a wire 106, and one or more probes 109a . . . 109c (collectively “probes 109”) extending from and/or partially nested in a probe housing 112, among other features and components as will be described. The enclosure 103 may include an outer surface and a hollow interior, and may further include one or more projections 115a, 115b (collectively “projections 115”) defining apertures or openings for insertion of zip-tie and/or wire-tie capability. For instance, the enclosure 103 of the sensor device 100 may be bound to rebar or other desirable location using zip-ties, wire ties, or other suitable connection mechanism via the apertures of the projections, as will be described.

Housed within the enclosure 105 of the sensor device 100 may be circuitry as well as additional components such as, for example, a temperature probe 118, a bulk material electrical resistivity probe 121, a pore solution electrical conductivity probe 124, and/or processing circuitry 127. The processing circuitry 127 may include, for example, a battery 130 or other power source, a microprocessor 133, impedance measurement circuitry 136, temperature measurement circuitry 139, a transceiver 142 (or a transmitter), a power switch 145, memory 148 (e.g., memory of the microprocessor 133 and/or a removable memory card), among other components as may be appreciated.

The impedance measurement circuitry 136 and/or the temperature measurement circuitry 139 may be part of or communicatively coupled to the processing circuitry 127, which includes a microprocessor 133 in various embodiments. The impedance measurement circuitry 136, the temperature measurement circuitry 139, and/or the processing circuitry 127 may be communicatively coupled to the wire 106, which may include an electrically conductive wire extending out of the enclosure 103 to connect to one or more probes 109. The probes 109 may include, for example, a pair of electrical resistivity and electrical conductivity probes 109a, 109b and a temperature probe 109c in some embodiments, although other probes 109 may be employed. In some embodiments, the electrical resistivity and electrical conductivity probes 109a, 109b may be gold-coated, although other suitable materials may be employed.

As may be appreciated, the electrical resistivity and electrical conductivity probes 109a, 109b may be used for electrical impedance measurement as they have a standard predetermined geometric factor value (e.g., a K-value) to be utilized for bulk material electrical resistivity and electrical conductivity of pore solution calculations, or other types of calculations.

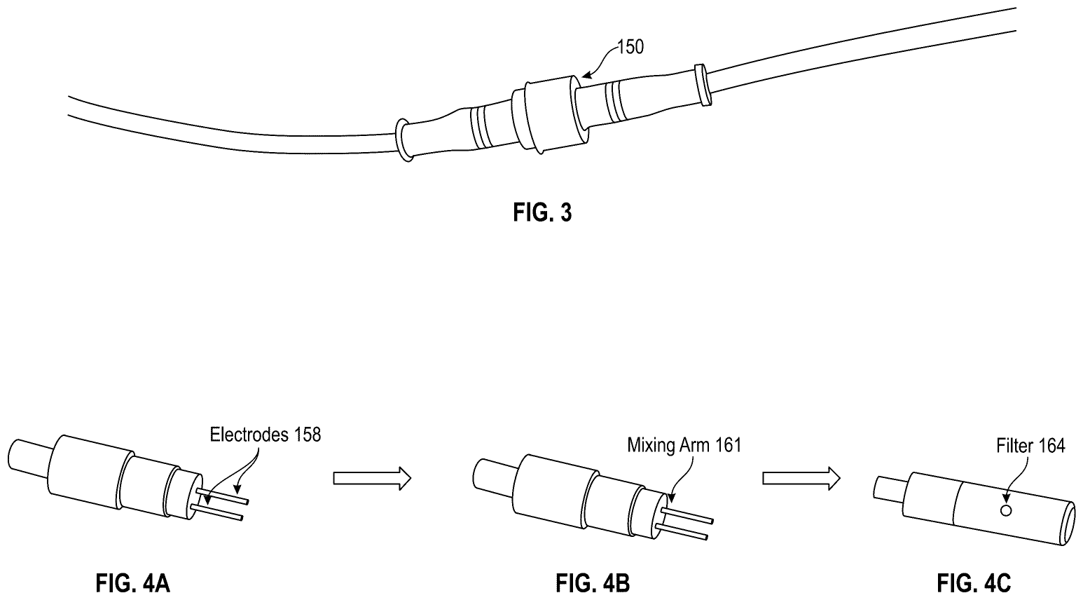

In some embodiments, the temperature and electrical resistivity probes 109a, 109b may be connected using an electrical connector, such as a four-pin waterproof electrical connector 150 to the sensor device 100 shown in FIG. 3. This connector may assist in reuse of the sensor device 100. For instance, the connector 150 of FIG. 3 may be used for precast concrete applications with probes 109 that are disposable or reusable, such that the connector 150 is positioned at least partially external to precast concrete. The connector 150 may couple to the wires 106, 152 of FIG. 1 or 2.

While the embodiment of FIG. 1 shows various types of probes 109, additional types of probes may be employed. For instance, in FIG. 2, the sensor device 100 includes not only a first wire 106 that couples the enclosure 103 and the components therein to a first probe arrangement (comprising the probes 109), the sensor device 100 further includes a second wire 152 that couples the housing and the components therein to a second probe arrangement.

In some embodiments, as shown in FIGS. 3A-3C, in order to monitor the electrical conductivity of a pore solution, the second probe arrangement may include electrodes 158 (FIG. 3A), a mixing arm 161 (FIG. 3B) (e.g., a plastic mixing arm 161), and a filter 164 (FIG. 3C) that is connected to the sensor device 100. The electrode 158 may include copper electrodes, the mixing arm 161 may include a plastic mixing arm, and/or the filter 164 may include a porous plastic filter in various embodiments. The filter 164 is not shown in FIGS. 3A and 3B for explanatory purposes. The working of the probes inside the pore solution conductivity relies on the same principles as that of electrical resistivity probe described herein.

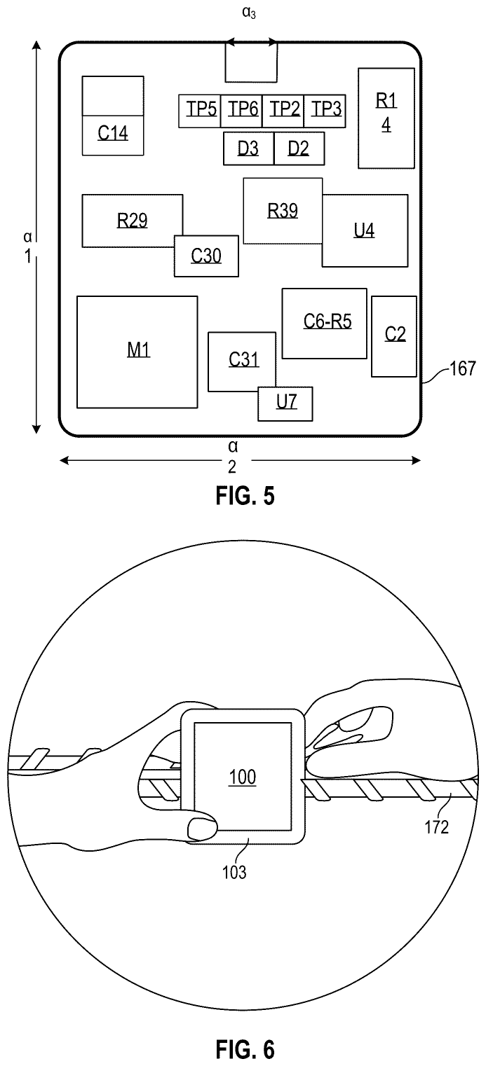

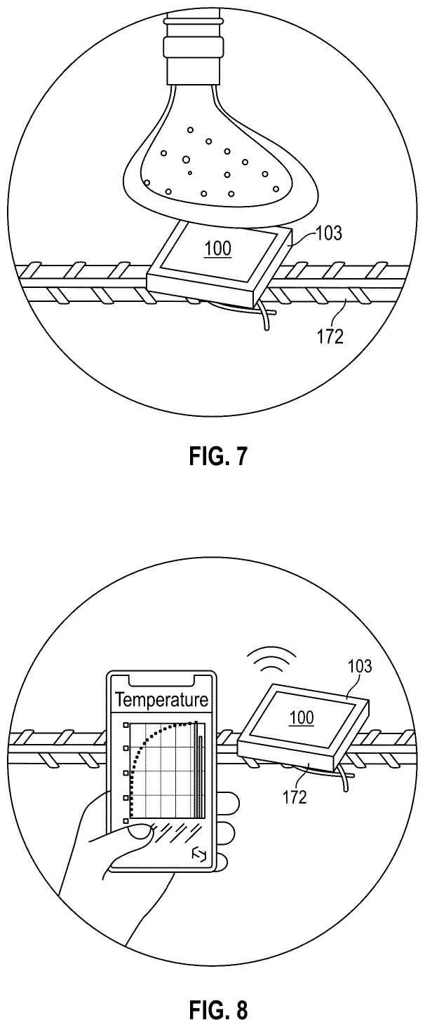

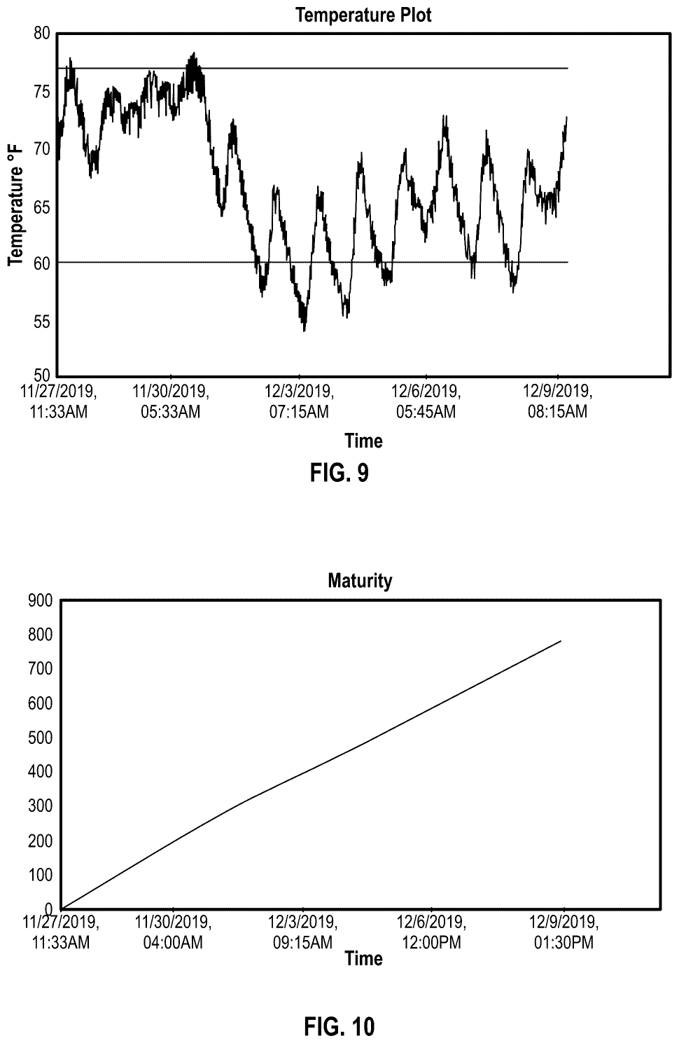

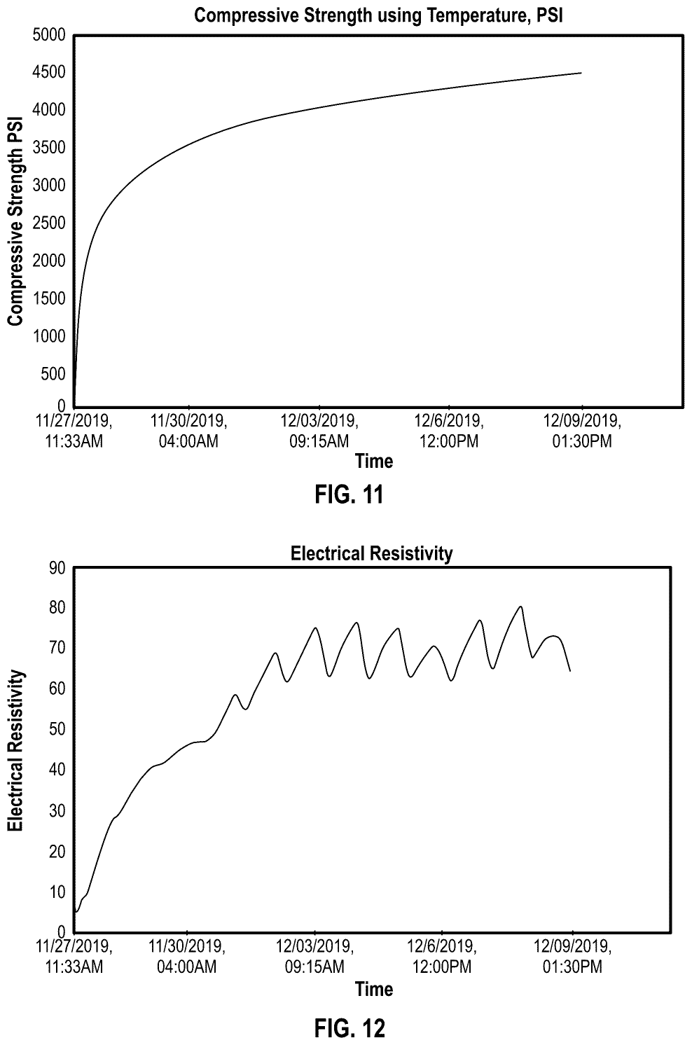

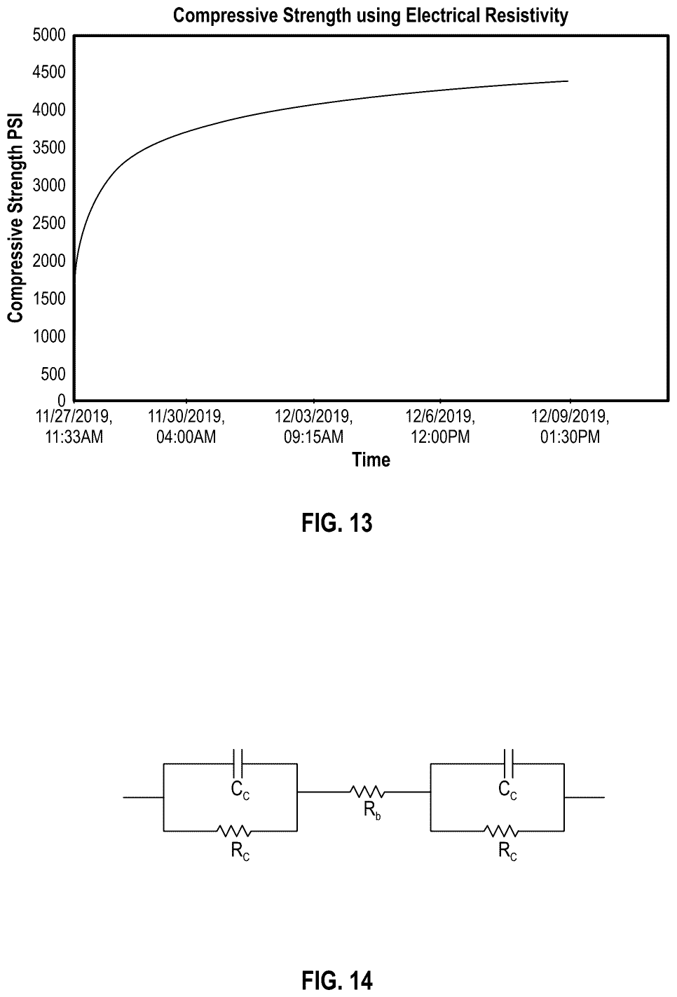

Turning now to FIG. 5, FIG. 5 is an interior layout of a printed circuit board (PCB) 167 that may be a part of the processing circuitry 127 is shown according to various embodiments of the present disclosure. In FIG. 5, the PCB 167 includes testpoints TP2, TP3, TP5, TP6, an antenna M1 (e.g., a Bluetooth chip antenna), diodes D2, D3 (e.g., TVS DIODE 5V 12.3V SOD923), capacitor C14 (e.g., CAP CER 10UF 10V X7R 0805), resistor R14 (e.g., RES SMD 100K OHM), resistor R29 (e.g., RES SMD 100K OHM 1%/16 W), capacitor C30 (e.g., CAP CER 01UF 25V X7R 0402), resistor R39 (e.g., RES SMD 47K OHM 1%/16 W), a network analyzer U4 (e.g., IC network analyzer), capacitor C31 (e.g., CAP CER 0.1UF 25V X7R 0402), capacitor-resistor C6-R5 (e.g., CAP CER 01 UF 25V), capacitor C2 (e.g., CAP CER 10 PF 10% 25V NPO 0402), and/or other components not described herein. The dimensions of α₁ may include 38.00 mm, α₂ may include 38.00 mm, and as may include 6.00 mm, although other desirable dimensions may be employed.

According to various embodiments described herein, the sensor device 100 and the processing circuitry 127 thereof, and/or an external computing device in communication with the sensor device 100, may be configured to determine initial and final setting times of concrete in a field compared to laboratory calibration; verify water to cement ratio of wet concrete and moisture content of solid concrete; determine in-situ compressive strength of concrete in real-time right from pouring through a twelve month period; make real-time predictions of compressive concrete strength and quality at predefined intervals (e.g., one-day, three-day, seven-day, twenty-eight-day, and fifty-six-day intervals) using electrical resistivity correlation; perform real-time detection of voids or soil contamination in wet concrete during and after the placement of concrete; quantify the properties of concrete, such as permeability, porosity, and diffusion using correlations to electrical resistivity measurements; quantify the pore solution electrical conductivity in concrete during and after placement; perform real-time detection of cracks in concrete structures and trace development over time; perform real-time determination of presence of chloride, sulphate, and/or other ions that affect the integrity and quality of concrete; determine the rate of corrosion of rebar inside the concrete using the absolute value of electrical resistivity of concrete; determine an absolute value of electrical resistivity from electrical impedance measurements and predicting the quality and strength parameters of concrete in real time for up to twelve months or other suitable period of time; perform real-time temperature and alternating current electrical impedance measurements of concrete for a period of up to twelve months or other suitable period of time; transmit or otherwise communicate the absolute value of quality and strength parameters of concrete and the change in parameters over time; perform temperature and alternating current electrical impedance measurement in concrete at a predefined period of time (e.g., twelve months) from a pour of concrete; transmit or otherwise communicate the time, temperature, and electrical impedance measurements to a remote server that processes the electrical impedance data to calculate the value of electrical resistivity of the concrete; determine quality and strength parameters of concrete; and/or transmit or otherwise communicate the quality and strength parameters in real-time to a predetermined enterprise to provide visual interpretation.

Now, a non-limiting example of a method of use of the above-described sensor is described. First, as shown in FIG. 6, a sensor device 100, as described herein, may be turned on or otherwise enabled and placed onto a concrete reinforcement structure 172, such as rebar. In order to maximize wireless antenna range (e.g., the range of the transceiver 142), the enclosure 103 and the components residing therein (e.g., processing circuitry 127) may be placed at a predefined distance from a surface of the concrete or other material to be poured. In some embodiments, the predefined distance includes approximately three inches (7.5 cm) or other suitable distance.

The sensor device 100 may be tightened or otherwise adequately coupled to the concrete reinforcement structure 172 (e.g., rebar) to reduce the risk of the sensor device 100 being flipped over when the concrete is poured. The transceiver 142 of the sensor device 100 (or other suitable receiver and/or transmitter) may be kept in an upward position to enable a connection and communication between the sensor device 100 and an external computing device, such as a mobile computing device (e.g., a smartphone, laptop, tablet, etc.). In some embodiments, the connection and communication are performed wirelessly although, in alternative embodiments, a third wire (not shown) may be positioned in the concrete such that the third wire projects from a cured surface of the concrete, allowing for a physical connecting between the third wire and one or more computing devices.

Second, before pouring concrete, the wire 110 that connects the enclosure 105 (and the components therein) to the probes 115 may be positioned under the concrete reinforcement structure 172 to protect it from potential damage. Then, it is ensured that temperature and resistivity probes 115 do not touch the ground or any other surface other than the fresh concrete mix. Finally, as shown in FIG. 5, the concrete is poured and cured, while the sensor device 100 starts collecting data which is stored in the memory 148 of the sensor device 100.

After the sensor has been properly installed and fresh concrete poured, a user can connect to the sensor using a computing device 175, for example, to search for nearby sensor devices 100 using a client application. In embodiment in which wireless communication is employed, the computing device 175 may pair with or otherwise connected to the sensor device 100 using the ZigBee® communication protocol, the Bluetooth® communication protocol, or other suitable near field communication protocol.

In some embodiments, the sensor device 100 may transmit data up to forty-nine feet (fifteen meters) of distance between the location of the sensor device 100 and the computing device 175. However, other desirable ranges may be employed. The frequency of the measurements can be adjusted to a desired time interval (e.g., every five minutes), and may have a life of the battery 130 (or a battery life) of six months after installation in some embodiments. It is understood that increasing the time between measurements may increase the life of the battery 130. The sensor device 100 thus may provide real-time data measurements on temperature, maturity, resistivity, and strength of concrete through metrics, graphs, or other data that may be rendered in a user interface to be shown in a display as well as, for example, a point and time when the data was recorded.

The data collected from the sensor device 100 may allow an individual to make more informed decisions during and after the pouring of fresh concrete. For instance, a client application (executable on the computing device 175) may be configured to enable an end user to set thresholds for when the concrete has reached the desired strength, notably, without having to wait three to seven days before getting lab results. Alerts and notifications may inform the user when it is the appropriate time to remove formwork and other building assistance equipment. Over time, all the data collected from the different concrete mix-design and the data collected from the sensor embedded in the fresh concrete could have the potential to provide real-time feedback based on the property of concrete use, job site geographic location, and concrete curing process.

Impedance Model for Electrical Property Characterization: Equivalent Circuit. FIG. 14 depicts a non-limiting example of an equivalent circuit that represents electrical properties of a material (e.g., concrete) to characterize its performance with time. There are many difficulties associated with choosing a correct equivalent circuit. It is derisible, however, to connect the different elements in the circuit to different regions in the impedance data of the corresponding sample. Given the difficulties and uncertainties in establishing this connection, researchers tend to take a pragmatic approach and adopt a circuit which they believe to be most appropriate from their knowledge of the expected behavior of the material under study, and demonstrate that the results are consistent with the circuit used. With the sensor described herein, different possible equivalent circuits were analyzed to find an appropriate equivalent circuit to represent concrete, cementitious materials, and polymers.

Special Bulk Material-Resistance Only. In the equivalent circuit, the electrical contacts were connected in series, and both the electrical contacts were represented using a capacitor Cc and a resistor R_{c} connected in parallel, as shown in FIG. 12. The bulk material was represented by a resistor R_{b}. In other words, in the equivalent circuit, R_{b} is the resistance of the bulk material, and R_{c} and Cc are resistance and capacitance of the contacts, respectively. Both contacts are represented with the same resistance R_{c} and capacitance Cc in embodiments in which they are made in an identical manner and include identical materials. A total impedance of the equivalent circuit Z₁ can be represented as shown in eq. 1 below.

$${Z}_{1} ( σ ) = \frac{{R}_{b} ( σ )}{1 + {ω}^{2} {R}_{b}^{2} {C}_{b}^{2}} + \frac{2 {R}_{C} ( σ )}{1 + {ω}^{2} {R}_{C}^{2} {C}_{C}^{2}} - j \{ \frac{2 ω {R}_{C}^{2} {C}_{C}^{2} ( σ )}{1 + {ω}^{2} {R}_{C}^{2} {C}_{C}^{2}} + \frac{2 ω {R}_{b}^{2} {C}_{b}^{2} ( σ )}{1 + {ω}^{2} {R}_{b}^{2} {C}_{b}^{2}} \} . \qquad ( eq . 1 )$$

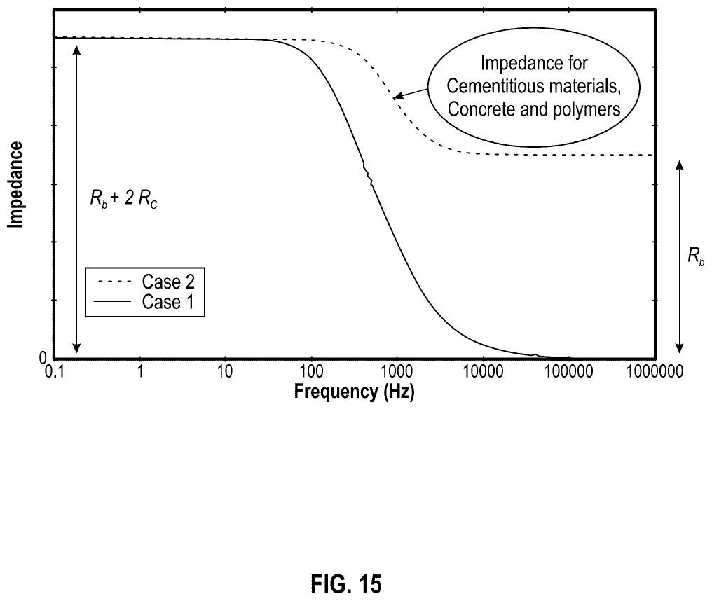

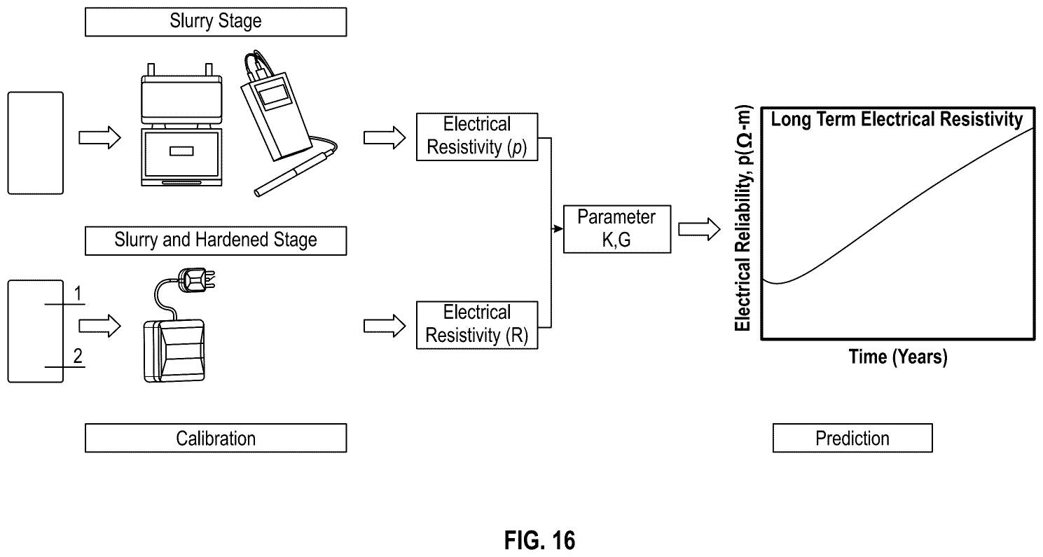

In this case, the capacitance of the bulk material, denoted C_{b}, is assumed to be negligible, as can be seen in FIG. 15. When the frequency of the applied signal is very low, ω→0, Z₂=R_{b}+2R_{c}, and when it is very high, ω→∞, Z₂=R_{b}. The shape of the curves shown in the figure is very much influenced by material response and the two probe instruments used for monitoring. Testing of concrete indicated that electrical impedance represented their behavior and hence the bulk material properties can be represented by resistivity and characterized at a frequency of 100 kHz to 1 MHz using the two probes. The electrical resistance of material can be obtained by measuring impedance at high AC frequencies and can be converted into electrical resistivity using K-value as shown below.

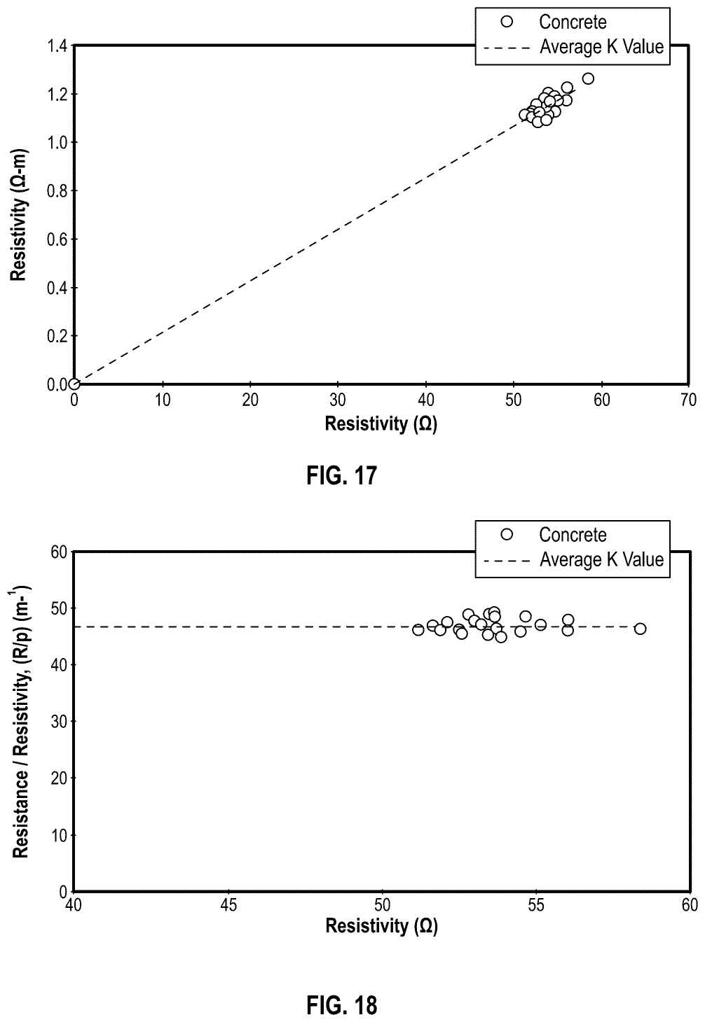

The relation between electrical resistance, electrical resistivity and K-value is given by eq. 1 below:

$$ρ = \frac{R}{K + GR} , \qquad ( eq . 2 )$$

where the constants K and G are constants. FIG. 17 further shows that electrical resistance and electrical resistivity have a linear relationship for concrete.

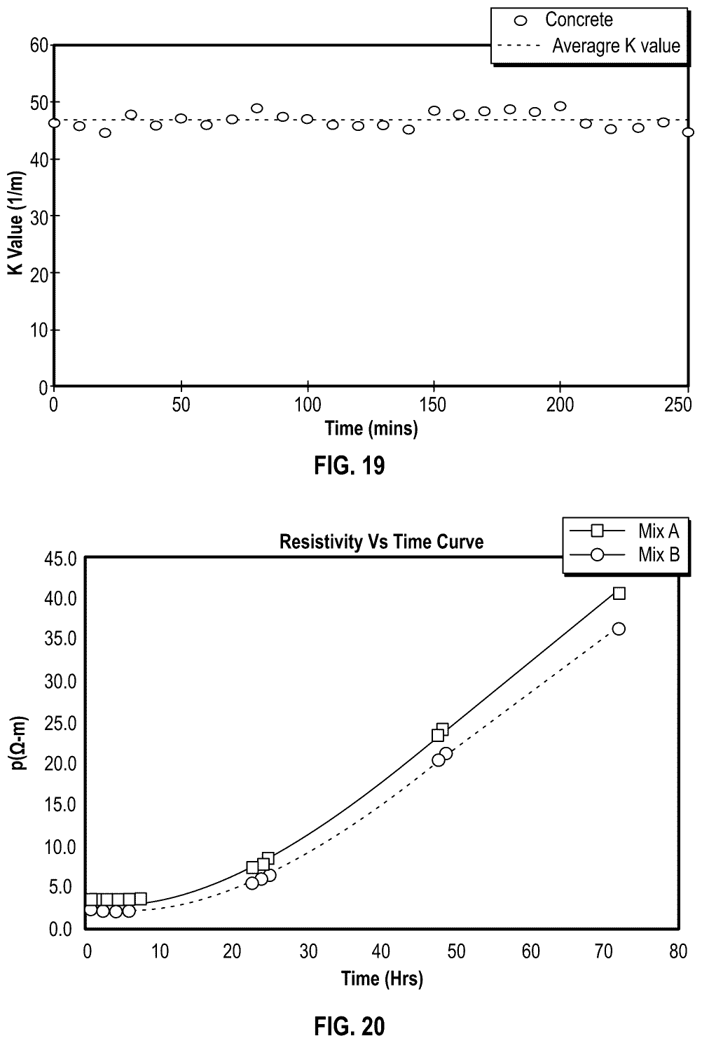

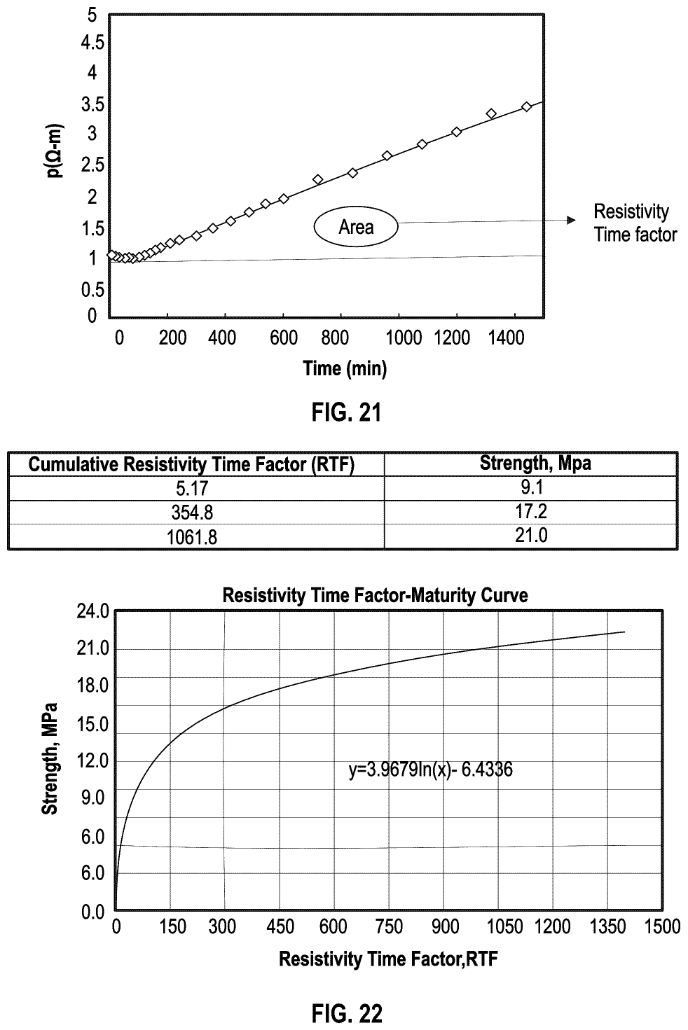

K-value Characterization. The electrical resistance R of the concrete was measured using the sensor described herein and the electrical resistivity p of the concrete was measured using both digital resistivity meter and conductivity meter for initial curing period to calculate the K-value at room temperature. The average K-value for cementitious materials for the prescribed sensor is developed during the initial curing period, as shown in FIG. 19.

The electrical impedance of concrete at certain ranges of frequency correlates to material property, electrical resistivity of concrete. Therefore, according to various embodiments, the electrical properties have been well correlated with important early-stage properties of concrete such that a variety of properties may be established including water cement ratio, in-situ compressive strength, initial and final setting times, transport property detection, pore solution electrical conductivity, hydration of cementitious materials and damage detection.

Determination of Water to Cement Ratio of Concrete. The measurement of water to cement ratio of concrete before or during pouring the concrete is important in the construction industry to ensure the appropriate quality of the concrete delivered by concrete trucks to the construction site. Every concrete mix design has a specified water to cement ratio. The water to cement ratio has an impact on the performance of concrete, its hydration, and porosity. Higher water content increases the porosity of the hardened concrete and thus, decreases its strength and durability. It is important to monitor the water/cement ratio in real-time. Accordingly, the sensor device 100 described herein helps avoiding pouring low-strength or low-quality concrete, the replacement of which will be very costly and, in some cases, impossible.

Findings. The electrical resistivity of the concrete slurry was found to vary between 2.5 to 3 Ω-m based on the water content added. For Mix A, the resistivity was 2.9 Ω-m, for Mix B it was 2.60 Ω-m, due to increased water to binder ration, as shown in FIG. 20. The resistivity of the flyash concrete varied between 36.13 to 40.5 for percentages Mixes A and B after three days of curing. It was also observed that the resistivity of flyash concrete were lower for higher water to binder ratio. For Mix A, the resistivity time factor was 51.7 Ω-m-day and compressive strength was 9.1 MPa after one day of curing. The resistivity time factor was 354.8 Ω-m-day and 17.2 MPa after two days of curing. After three days of curing, the resistivity time factor was 1061.8 MPa and compressive strength of 21 MPa. Accordingly, it is evident that the water cement ratio impact shows correlation to electrical resistivity measurements during initial placement of concrete. Such electrical measurements made in the field provide indication of quality of cementitious materials without requiring additional testing.

Prediction of In-Situ Compressive Strength of Concrete after Pouring. Assessment of the compressive strength of concrete during the first few days from pouring up to sever days after pouring is important for the optimization of formwork removal, post tensioning, and opening of roadways especially in the winter. The rate of hydration of every concrete mix design differs due to various factors of influence. The electrical resistivity of concrete over time can be used to estimate the compressive strength of concrete, rate of hydration, and calculation of percentage of strength gain.

Long term strength prediction may further be employed. Specifically, established resistivity time factor (RTF) versus strength correlation may be employed to predict the long-term compressive strength of concrete. A depiction of the results is presented below.

One-Day Results. The RTF Factor for one day was 33.7 Ω-m-day. Using a prediction curve, the compressive strength was 7±0.5 MPa. The actual one-day strength obtained by performing compressive strength was 7.03 MPa, as shown in FIG. 22.

Two-Days Results. The RTF Factor for two days was 286.2 Ω-m-day. Using a prediction curve, the compressive strength was 15.4±0.5 MPa. The actual two-day strength obtained by performing compressive strength was 15.3 MPa.

Three-Days Results. The RTF Factor for 3 days was 913.3 Ω-m-day. Using a prediction curve, the compressive strength was 19.5±0.5 MPa. The actual one-day strength obtained by performing compressive strength was 19.1 MPa. The prediction of compressive strength using resistivity time factor curve was accurate up to about ±0.5 MPa. Accordingly, it is evident that the compressive strength shows linear correlation to electrical resistivity and resistivity time factor (RTF) may be utilized for prediction of compressive strength without need for break tests.

Initial and Final Setting of Concrete (ASTM C403). Currently, the determination of initial and final setting time concrete is performed using Vicat Apparatus ASTM C403: “Standard Test Method for Time of Setting of Concrete Mixtures by Penetration Resistance.” This laboratory procedure is labor intensive and cannot be employed in the field. Setting times are important for deciding on the surface finishing of concrete and for other sequence of operations in the construction. Monitoring the absolute value of electrical resistivity of concrete enables identification of different stages of hydration reaction inside concrete leading to correlation to setting time of concrete. Accordingly, electrical impedance measurements present a non-invasive, in-site process for detection of setting time of concrete.

Assessment of Transport properties of Concrete. Two important electrical parameters required for reliable and accurate assessment of the microstructural properties of concrete are electrical conductivity of bulk concrete and its pore solution. The measurement of these two properties also enables calculation of formation factor for concrete materials over time. One of the major drawbacks of current available techniques is the inability to monitor these electrical properties of concrete materials on the field and in real time for long term. The measurement of bulk electrical conductivity of concrete has been demonstrated. Monitoring the electrical conductivity in real time enables determination of transport properties of concrete. Accordingly, this assessment can substitute Standards such as ASTM C12O2: “Standard Test Method for Electrical Indication of Concrete's Ability to Resist Chloride Ion Penetration” and ASTM C1543: “Standard Test Method for Determining the Penetration of Chloride Ion into Concrete by Ponding.”

Within the embodiments of the invention described, the electrical and temperature measurements may be made using disposable and/or reusable probes connected to sensor via a 4 pin waterproof electrical connector. For example, a disposable sensor may exploit Bluetooth connectivity for short range low power communications and ad-hoc, LORA or other network protocols to communicate electrical measurement data to a node or nodes wherein it is pushed to remote servers, what is commonly referred to today as “the cloud, through one or more different network interfaces and/or network protocols. Subsequently, this cloud stored data can be analyzed in real time and/or periodically to determine one or more of the measurements described.

In addition to measuring, for example, temperature, AC electrical resistivity, and AC electrical conductivity of pore solution, it would be evident that additional parameters as discussed and described in respect of embodiments of the invention may be measured and monitored, including, but not limited to, concrete moisture content, concrete internal relative humidity, concrete pH, concrete mixture consistency, concrete workability (slump), and concrete air content.

Accordingly, based on the foregoing, a method for performing real-time concrete monitoring is described that includes providing at least one sensor device 100 (e.g., one or more of the sensor device 100 described above). The sensor device 100 includes an enclosure 103 configured to couple the sensor to a concrete reinforcing structure 172, the enclosure comprising a temperature probe 118, impedance measurement circuitry 136, temperature measurement circuitry 139, and a transceiver 142. The sensor device 100 further includes a first electrical conducting wire 106 (or “first wire 106”) extending out from the enclosure 103 being electrically and/or communicatively connected to at least one probe 109. The processing circuitry 127 may be configured to perform at least one impedance measurement using the at least one probe 109 and a temperature measurement using the temperature probe 109c; and communicate at least one of the at least one impedance measurement, the at least one temperature measurement, and derivative information associated therewith, to a computing device 175 via the transceiver 142. The method further includes affixing the enclosure 103 of the at least one sensor device 100 to the concrete reinforcing structure 172; pouring concrete or other material in an area such that the at least one sensor device 100 is wholly or partially encapsulated in the concrete such that the at least one probe 109 is in contact with the concrete; receiving, by at least one computing device 175, at least one of the at least one impedance measurement, the at least one temperature measurement, and derivative information associated therewith from the at least one sensor device 100; determining, by the at least one computing device 175, at least one of a temperature, a maturity, a resistivity, and a strength of the concrete based on at least one of: the at least one impedance measurement, the at least one temperature measurement, and derivative information associated therewith; and displaying, by the at least one computing device 175, information associated with at least one of the temperature, the maturity, the resistivity, and the strength of the concrete in a display device.

Additionally, a method is described that includes performing an AC electrical impedance measurement using a sensor device 100 positioned inside a material comprising at least one of: concrete, cementitious material, liquid, soil, polymer, and a combination thereof in real-time; and determining a characteristic of the material based upon at least the electrical impedance measurement, wherein determining the characteristic of the material further comprises adjusting the AC electrical impedance measurement based at least in part on an appropriate electrical circuit, geometric factor, and temperature.

In some embodiments, the material includes concrete in one of a wet and a hardened state, and the characteristic of the material is at least one of the following: a water to cement ratio of the concrete; an estimated in-situ compressive strength of the concrete after pouring; at least one of seven-day, twenty-eight-day, and fifty-six-day compressive strength of the concrete; at least one of the initial and final setting time of the concrete; a transport property of the concrete (e.g., permeability, diffusivity, porosity, and any combination thereof); a presence of voids inside the concrete; a presence of a crack within the concrete; and pore solution characteristics.

The electrical impedance may be obtained using an equivalent circuit, as shown in FIG. 14, and a dependence of geometric factor determined to obtain at least one of electrical resistivity and electrical conductivity to determine the characteristic, where the characteristic may include a water to cement ratio of the concrete; an in-situ compressive strength of the concrete after pouring; a prediction of at least one of seven-day, twenty-eight-day, and fifty-six-day compressive strength of the concrete; a detection of at least one of the initial and final setting time of the concrete; and an assessment of a transport property of the concrete selected from a group consisting of permeability, diffusivity, and porosity.

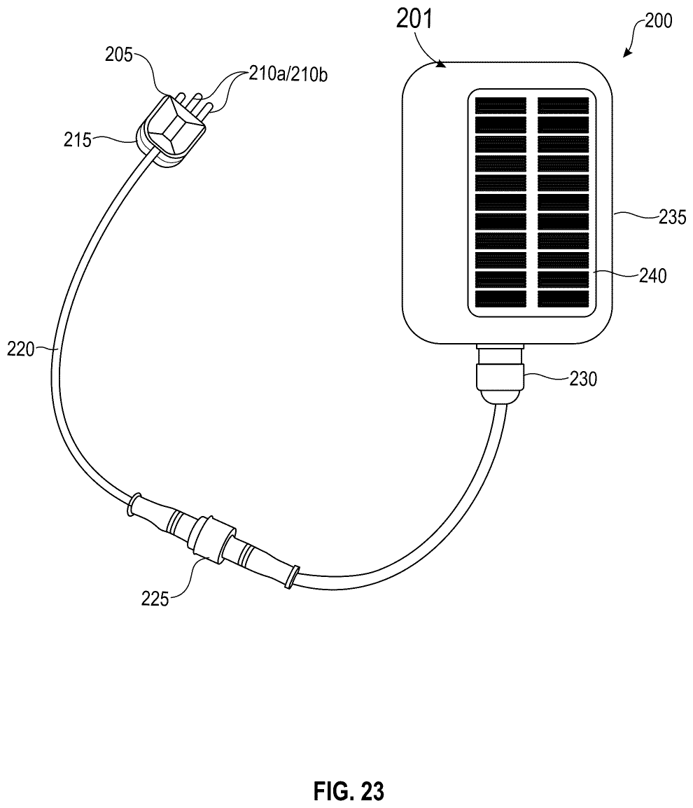

Referring now to FIGS. 23-30, various embodiments of a sensor system for real-time monitoring of concrete, soil, and other mediums are illustrated. The sensor system may comprise a sensor 200 and a gateway 310, among other components as will be described.

FIG. 23 shows an isometric view of the sensor 200, which may include a sensor node 201 and a sensor probe 220 that are detachably attachable to one another. The sensor node 201 may be housed within a sensor enclosure 235. In some aspects, a solar panel 240 may be positioned on or embedded in the top surface of the sensor enclosure 235 to provide power to the device or a battery (not shown) thereof. The sensor enclosure 235 may include one or more probe connection ports 230a . . . 230d (collectively “ports 230”), allowing for connection of the sensor probe 220 to the sensor node 201.

The sensor probe 220 may comprise a probe enclosure 215 at one end of a wire or cable. Within the probe enclosure 215, there may be a thermocouple 205 for temperature measurement and two conductive contact points 210a and 210b for electrical measurements. The contact points 210 may include pins in some embodiments. In some embodiments, the conductive contact points 210a, 210b may be gold coated metal contact points. The gold coated metal contact points serve as the electrodes for electrical impedance measurements in the sensor probe. Gold coating is utilized due to its excellent electrical conductivity and resistance to corrosion, ensuring accurate and consistent measurements over time. The use of gold coating also helps minimize electrode polarization effects, which can interfere with impedance measurements. These contact points are designed to make direct contact with the material being monitored, such as concrete or soil, allowing for precise measurement of electrical properties like resistivity and conductivity. The gold coating provides a stable interface between the electrode and the surrounding medium, reducing the likelihood of chemical reactions that could affect measurement accuracy. Additionally, the durability of gold-coated contact points makes them suitable for long-term deployment in harsh environments often encountered in construction and geotechnical applications. The cable of the sensor probe 220 may connect to the sensor node 201 via a detachable connection 225, which may allow for easy replacement or swapping of the sensor probe 220 if needed.

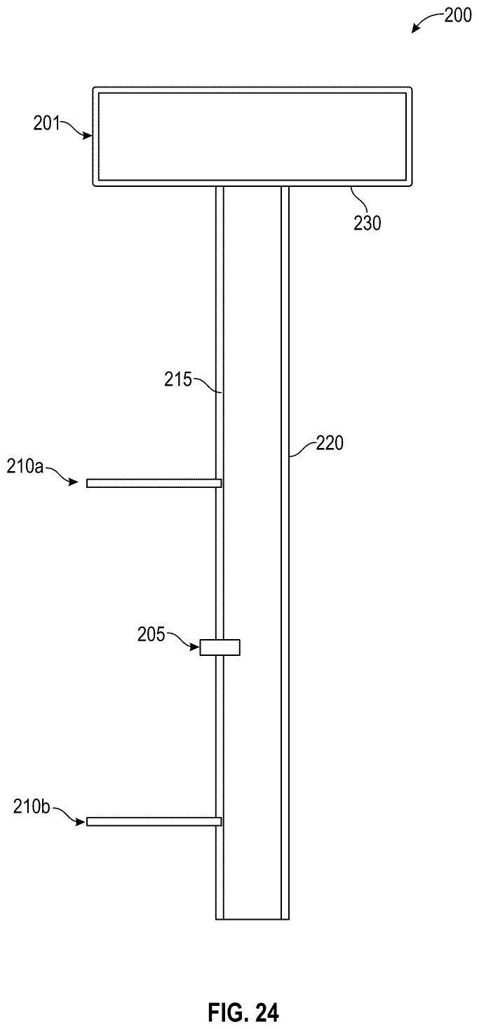

FIG. 24 presents a schematic diagram of the sensor 200, further illustrating the arrangement of components within the sensor probe 220. The probe enclosure 215 may house the sensing elements in a vertical arrangement, with a conductive pin 210a at the top, the thermocouple 205 in the middle, and another conductive pin 210b at the bottom.

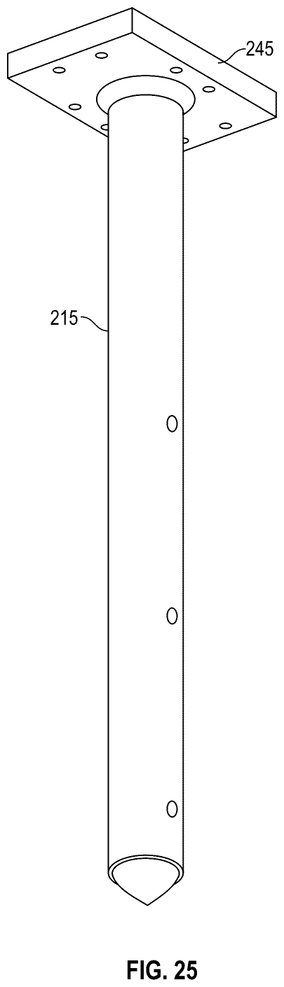

FIG. 25 provides an orthogonal view of the sensor probe 220, showing the probe enclosure 215 and a probe connection point 245. The probe enclosure 215 may be an elongated cylindrical structure designed to be embedded in the material being monitored. The probe connection point 245 may serve as an interface between the probe enclosure 215 and the main sensor unit.

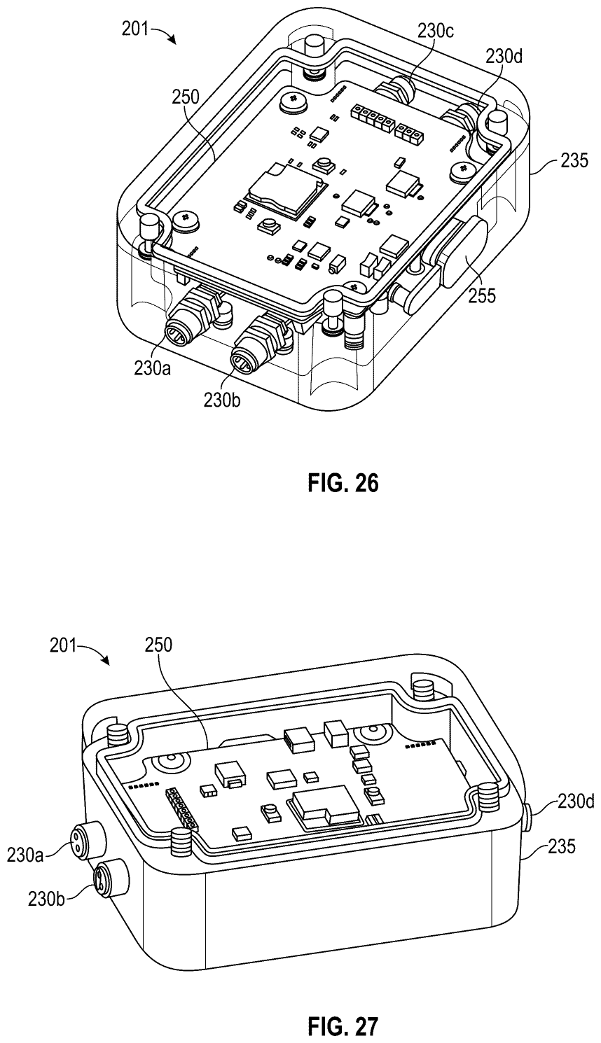

FIGS. 26 and 27 illustrate isometric views of the sensor node 201. The sensor node 201 may be housed within a sensor enclosure 235 and may contain a printed circuit board (PCB) 250 that holds various electronic components. The sensor node 201 may feature multiple probe connection ports, labeled as 230a, 230b, 230c, and 230d, positioned around the perimeter of the sensor enclosure 235. A charging port 255 may be located on one side of the sensor enclosure 235, providing a means to recharge the internal battery or power the device.

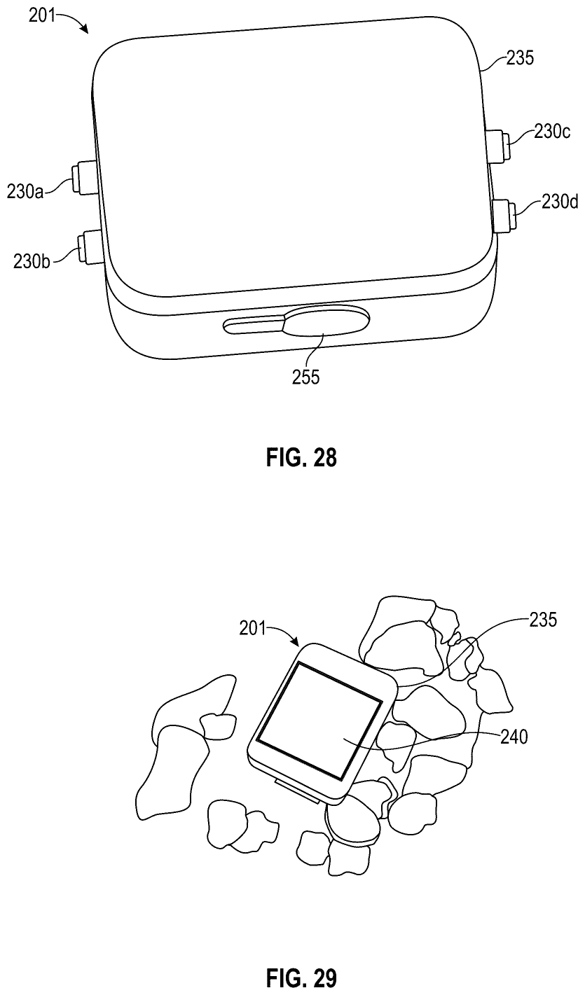

FIG. 28 presents another isometric view of the sensor node 201, further illustrating the arrangement of the probe connection ports 230a, 230b, 230c, and 230d around the perimeter of the sensor enclosure 235. The charging port 255 may be visible on the front face of the sensor enclosure 235.

FIG. 29 shows the sensor node 201 in an outdoor environment, demonstrating its ability to operate in various environmental conditions. The sensor enclosure 235 may be placed on a rocky surface surrounded by grass, illustrating its rugged design suitable for field deployment.

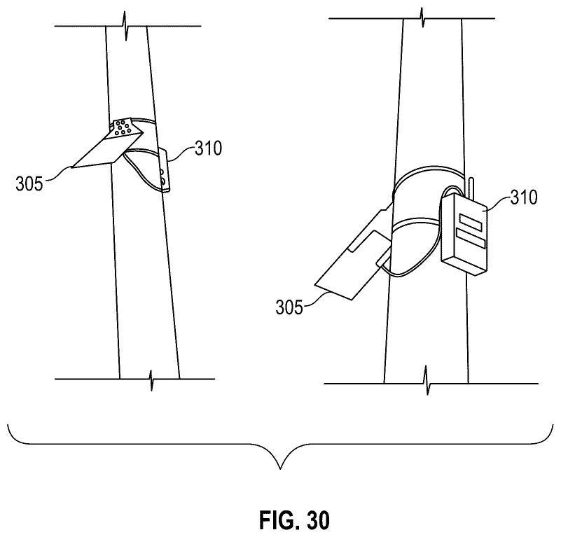

FIG. 30 illustrates two perspective views of a gateway 310 and a solar panel 305 that may supply power to the gateway. The system may be mounted on utility poles in different configurations. The solar panel 305 may be a flat, rectangular structure attached to the utility pole, positioned at an angle to maximize exposure to sunlight for energy harvesting. The gateway 310 may be a compact, box-shaped enclosure containing sensor electronics and communication components. The arrangement of the solar panel 305 and gateway 310 may allow for flexible installation options depending on specific site requirements and optimal positioning for solar energy collection and wireless communication.

The sensor system illustrated in FIGS. 23-30 may provide a comprehensive solution for real-time monitoring of concrete, soil, and other mediums. The modular design of the sensor 200, with its detachable sensor probe 220, may allow for easy maintenance and replacement of components. The sensor node 201 may provide multiple connection options for probes and include integrated power management features. The gateway 310, powered by a solar panel 305, may enable long-term autonomous operation in outdoor environments. This system may be capable of collecting and transmitting data on temperature, electrical properties, and other parameters of the monitored material, providing valuable insights for construction, geotechnical, and environmental applications.

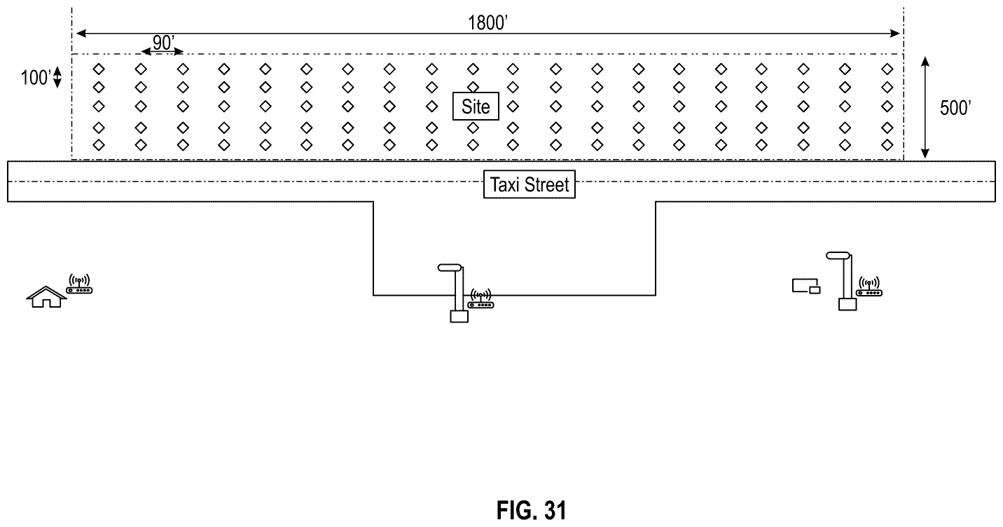

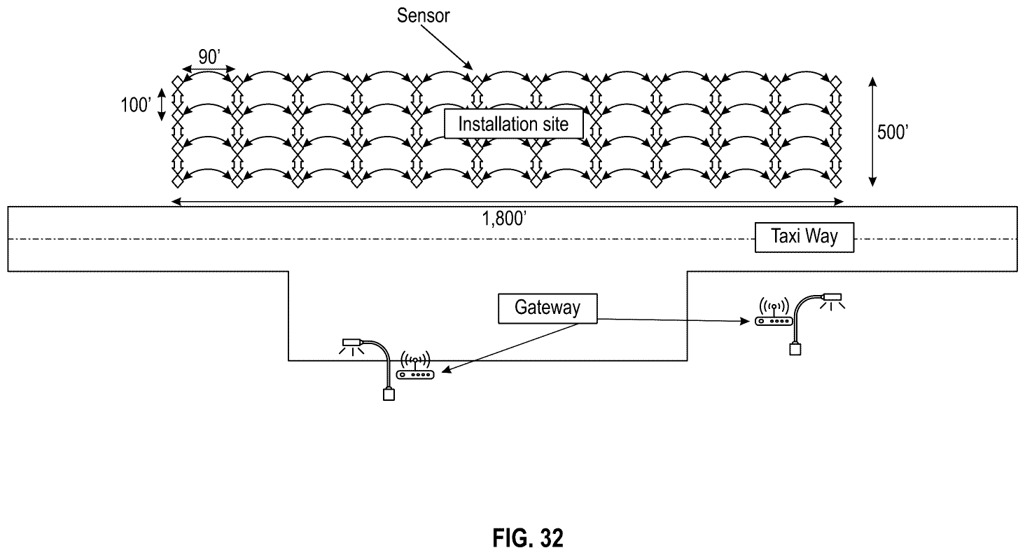

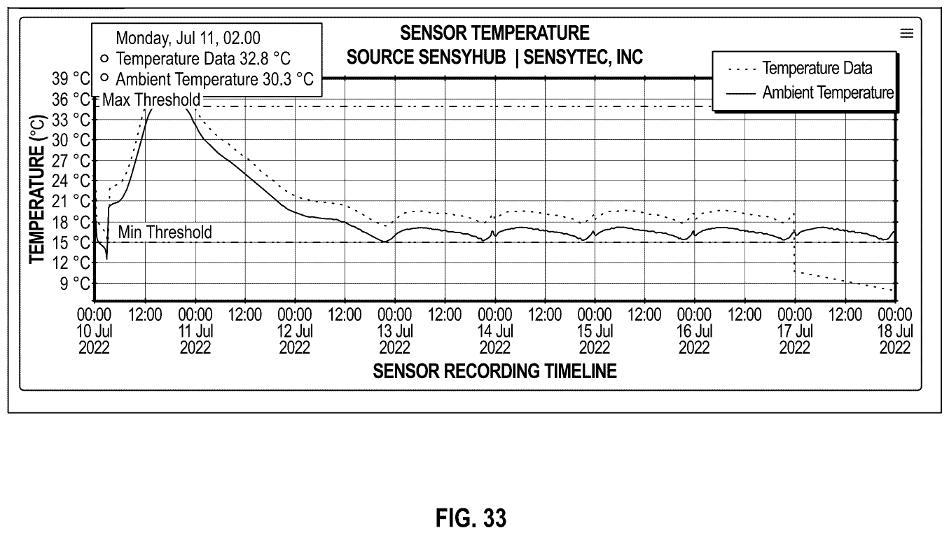

Referring again to FIGS. 31-32, a sensor system for long-term monitoring using high-frequency electrical impedance is illustrated. The system may be designed for continuous monitoring of parameters over extended durations, overcoming limitations of traditional monitoring systems that often provide intermittent data or require manual intervention.

As shown in FIG. 31, the system may include a multitude of sensors 200 embedded within an installation site, which can include concrete, soil, or other desired medium. A grid of diamond-shaped markers, each representing a sensor 200, may be distributed evenly throughout the installation site, representing the locations of embedded sensors 200 within the installation side.

The sensor 200 may be equipped with probes for measuring temperature and electrical resistivity using high-frequency electrical impedance techniques. These measurements may allow for detailed analysis of the physical and chemical properties of the concrete, soil, or other medium, providing insights into parameters such as moisture content, structural integrity, and potential contamination.

FIG. 32 illustrates a system diagram of the wireless sensor network for real-time monitoring. Within this installation site, a grid of sensors 200 may be arranged in a 4×13 pattern, depicted by diamond-shaped markers connected by blue lines. Each sensor 200 can communicate to one another and may form a mesh network in some implementations.

Adjacent to the installation site, a vertical strip labeled “Task-Way” may represent a designated path for maintenance or data collection activities. To the right of the task-way, a section labeled “Gateway” may house the central communication and data processing unit for the sensor network.

The sensor arrangement may allow for comprehensive monitoring of the concrete structure, with sensors 200 placed at regular intervals throughout the installation site. This configuration may enable the system to collect data from multiple points, providing a detailed view of the concrete's condition and performance in real-time.

The system may utilize long range communication technology, such as LoRa, LoRaWAN, NB-IoT, LTE-M, WiFi or Sigfox, for wireless data transmission to the central gateway. These technologies may offer benefits such as low power consumption, long-range coverage, and robust security features, making them suitable for applications in remote or challenging environments.

Each sensor 200 may incorporate a solar panel 240 for autonomous recharge capability, ensuring continuous operation without reliance on external power sources. This feature may address the challenge of power supply in remote or off-grid locations, enhancing the sustainability and reliability of the monitoring system.

The gateway 310, which can be powered by a solar panel 305 and/or battery, may communicate with the sensors 200, collecting data and sending it for processing. The data processing and visualization component may include digital twin technology for real-time visualization of the concrete structure, facilitating informed decision-making and proactive management.

The wireless sensor system may find applications in diverse fields such as agriculture, environmental monitoring, infrastructure management, and industrial process control. For example, in the context of infrastructure monitoring, the system may be used to evaluate the structural health of buildings, bridges, and dams by measuring concrete integrity and moisture ingress.

Future developments of the system may include integration of edge computing capabilities to perform data analytics at the sensor node level, implementation of artificial intelligence and machine learning algorithms for predictive analytics, exploration of blockchain-based solutions for data integrity, and leveraging 5G networks to enhance data transmission speeds and support real-time applications.

The comprehensive system illustrated in FIGS. 31-32 may represent a transformative approach to long-term monitoring, emphasizing the potential to revolutionize environmental sustainability, industrial automation, and urban management practices through continuous, real-time data collection and analysis.

Accordingly, the present disclosure describes a thermo-electrical wireless sensor system using high-frequency electrical impedance, with solar recharge capability for long-term monitoring. The thermos electrical sensor system comprises a single or plurality of sensors 200 communicating with a single or plurality of gateways 310. A sensor 200 comprises a sensor node 201 and a single or plurality of probes 220 as shown in FIG. 23.

Each sensor node 201 within the system can include temperature measurement circuitry to measure ambient temperature and temperature inside any material with high accuracy using the thermocouple 205 and/or the conductive contact points 210.

The sensor node 201 can further include impedance measurement circuitry that utilizes a high-frequency electrical impedance techniques to measure electrical resistivity of the surrounding medium/materials using the thermocouple 205 and/or the conductive contact points 210. High-frequency impedance measurement allows for detailed analysis of materials and environmental conditions. This provides insights into soil moisture, structural integrity of materials, or fluid characteristics, depending on the application.

According to various embodiments, the sensor probe 220 is a small component responsible for holding temperature thermocouples 205 and conductive contact points 210 (e.g., gold-plated metal contacts) for electrical impedance measurement. The thermocouple 205 and/or the conductive contact points 210 can connected to the sensor node 201 via thin wires. The temperature thermocouple 205 connects to the temperature measurement circuitry and the conductive contact points 210 can connect to the impedance measurement circuitry, which can be embodied in the printed circuit board 250. The design of the sensor probe 201 leverages high-frequency electrical impedance. The probe 220 can be attached to the sensor node 201 through a wire that directly connects to the sensor node 201 through the probe connection port 230 or it can be attached to a detachable connection making it replaceable for applications as shown in FIG. 23. The probe 220 can be a wire with an enclosure holding the thermocouple 205 and conductive contact points 210 (e.g., gold-plated metal contacts) as shown in FIG. 23 or a tube holding the thermocouple 205 and the conductive contact points 210 or in any other form holding the thermocouple 205 and the conductive contact points 210.

Sensor nodes 201 can include long range communication modules (not shown) configured to operate in the ISM bands (e.g., 868 MHz, 915 MHZ, or 2.4 GHZ), ensuring robust and long-range communication capabilities. Long Range modulation techniques, such as Chirp Spread Spectrum (CSS), optimize data transmission efficiency and reliability over varying environmental conditions. Long Range modules also have Low power consumption suitable for battery-operated devices.

Sensor nodes 201 can include a microcontroller unit (MCU) and/or a central processing unit (CPU) for the sensor system. The MCU handles data acquisition, processing, and communication. It also is configured to manage power usage to extend battery life.

Each sensor node 201 can incorporate a solar panel 240 for harvesting solar energy. Photovoltaic cells of the solar panel 240 convert sunlight into electrical energy, which is stored in rechargeable batteries within the sensor node 201. Solar recharge capability ensures continuous operation and reduces the need for manual intervention or battery replacements, making the system suitable for remote and off-grid deployments. Mounted atop the sensor node 201, the solar panel 240 harnesses solar energy to recharge the system's internal battery, enabling continuous operation as shown in FIG. 29. The solar recharge system ensures the battery is charged during daylight hours.

To this end, each sensor node 201 can include a rechargeable battery and power regulation circuitry. The power regulation circuitry enables a stable power supply to the sensor system and manages charging and discharging cycles to maximize battery lifespan.

Each sensor node 201 can include a rugged and weatherproof enclosure 235 housing the core components of the system, including the wireless communication module and the thermo-electrical measurement system, among other components described herein. The weatherproof casing protects the sensor and electronic components and is designed to withstand harsh environmental conditions.

Data collected by sensor nodes 201 can be transmitted wirelessly to a central gateway 310 or base station using wireless communication technology. At the central location, data is processed and transmitted to a server (not shown) where it is analyzed, and visualized through a graphical user interface, which is rendered on a display of a client computing device. Analytical tools and algorithms detect trends, anomalies, or critical thresholds, enabling stakeholders to make timely decisions and implement proactive measures.

The gateway 310 include a base station housing wireless communication circuitry, cellular communication circuitry, a microcontroller, power management circuitry, and/or a rechargeable battery. The gateway 310 can be connected to a solar panel 305 to enable continuous operation and reduce the need for manual intervention or battery replacements, making the system suitable for remote and off-grid deployments. The gateway 310 uses cellular communication to transfer processed data from sensor nodes 201 to the server.

The thermo-electrical sensor can be configured to measure, collect, and send temperature and impedance data at predefined intervals. The high-frequency impedance measurements provide insights into various environmental parameters.

The MCU can be configured to process raw data from the sensors and implements algorithms to analyze and interpret impedance measurements. Processed data is transmitted to a remote server via the wireless communication gateway. This continuous communication system enables real-time monitoring and analysis.

According to various embodiments described herein, the sensor node 201 and the processing circuitry thereof, and/or an external computing device in communication with the sensor node 201 may be configured to determine a variety of parameters for materials as stated below. The thermo-electrical wireless sensor system can monitor a variety of materials by leveraging high-frequency electrical impedance measurements and temperature data. For concrete and building materials, the system can be used for long term structural integrity, moisture content, and temperature. The system can determine initial and final setting times of concrete in a field compared to laboratory calibration; verify water to cement ratio of wet concrete and moisture content of solid concrete; determine in-situ compressive strength of concrete in real-time right from pouring through a twelve month period; make real-time predictions of compressive concrete strength and quality at predefined intervals (e.g., one-day, three-day, seven-day, twenty-eight-day, and fifty-six-day intervals) using electrical resistivity correlation; perform real-time detection of voids or soil contamination in wet concrete during and after the placement of concrete; quantify the properties of concrete, such as permeability, porosity, and diffusion using correlations to electrical resistivity measurements; quantify the pore solution electrical conductivity in concrete during and after placement; perform real-time detection of cracks in concrete structures and trace development over time; perform real-time determination of presence of chloride, sulphate, and/or other ions that affect the integrity and quality of concrete; determine the rate of corrosion of rebar inside the concrete using the absolute value of electrical resistivity of concrete; determine an absolute value of electrical resistivity from electrical impedance measurements and predicting the quality and strength parameters of concrete in real time for up to twelve months or other suitable period of time; perform real-time temperature and alternating current electrical impedance measurements of concrete for a period of up to twelve months or other suitable period of time; transmit or otherwise communicate the absolute value of quality and strength parameters of concrete and the change in parameters over time; perform temperature and alternating current electrical impedance measurement in concrete at a predefined period of time (e.g., twelve months) from a pour of concrete; transmit or otherwise communicate the time, temperature, and electrical impedance measurements to a remote server that processes the electrical impedance data to calculate the value of electrical resistivity of the concrete; determine quality and strength parameters of concrete; and/or transmit or otherwise communicate the quality and strength parameters in real-time to a predetermined enterprise to provide visual interpretation. The system can be used for monitoring the health of buildings, bridges, and other infrastructure for signs of deterioration or damage.

Now, a non-limiting example of a method of use of the above-described sensor is described. First a sensor device, as described herein, may be turned on or otherwise enabled and placed onto a concrete reinforcement structure, such as rebar.

The sensor node 201 may be tightened or otherwise adequately coupled to the concrete reinforcement structure (e.g., rebar) to reduce the risk of the sensor device being flipped over when the concrete is poured. The sensor node of the sensor device (or other suitable receiver and/or transmitter) may be kept in an upward position to enable a connection and communication between the sensor device and an external computing device, such as a gateway. The sensor node 201 can be configured to be placed outside the concrete with only the probe 220 encountering the material of interest.

Second, before pouring concrete, the wire that connects the enclosure 235 (and the components therein) to the probes 220 may be positioned under the concrete reinforcement structure to protect it from potential damage. Then, it is ensured that temperature and electrical impedance probes 220 do not touch a ground or any other surface other than the fresh concrete mix. Finally, the concrete is poured and cured, while the sensor node 201 starts collecting data.

The sensor node 201 can transmit data up to a gateway 305 up to one mile in distance, although other distances can be envisioned using intermediary relay devices. As such, other desirable ranges may be employed. The frequency of the measurements can be adjusted to a desired time interval (e.g., every five minutes), and may have a battery life of twenty years after installation in some embodiments. It is understood that increasing the time between measurements may increase the life of the battery. The sensor node 201 thus may provide real-time data measurements on temperature, maturity, resistivity, and strength of concrete through metrics, graphs, or other data that may be rendered in a user interface to be shown in a display as well as, for example, a point and time when the data was recorded.

The data collected from the sensor device may allow an individual to make more informed decisions during and after the pouring of fresh concrete. For instance, a client application may be configured to enable an end user to set thresholds for when the concrete has reached the desired strength, notably, without having to wait three to seven days before getting lab results. Alerts and notifications may inform the user when it is the appropriate time to remove formwork and other building assistance equipment. Over time, all the data collected from the different concrete mix-design and the data collected from the sensor embedded in the fresh concrete could have the potential to provide real-time feedback based on the property of concrete use, job sites geographic location, and concrete curing process.

The sensor system can be utilized for monitoring moisture content, salinity, temperature, compaction, and other properties of soils or rocks. This system also allows for agricultural monitoring to optimize irrigation and fertilization, and environmental monitoring to assess soil health.

The thermo-electrical wireless sensor system may be applied to monitor various materials beyond concrete and soil. For metals, the system may monitor corrosion, stress, and temperature in pipelines, storage tanks, and structural components to detect early signs of corrosion or mechanical failure. In wooden structures such as buildings and bridges, the system may track moisture content, temperature, and structural integrity to identify rot, termite infestations, and other forms of degradation. For polymers and plastics, the system may monitor degradation, temperature, and mechanical stress in industrial and consumer products to ensure longevity and performance.

In the field of ceramics and composites, the system may be used to detect cracking, monitor temperature, and measure mechanical stress in high-performance materials used in aerospace, automotive, and industrial applications. For water bodies, the system may monitor salinity, temperature, and contamination levels in lakes, rivers, and reservoirs to assess water quality and detect pollution. In food product monitoring, the system may track moisture content, temperature, and spoilage indicators to ensure quality and safety of perishable items during storage.

The system may also find applications in textile monitoring, where it can measure moisture content, temperature, and degradation to assess fabric quality and performance in clothing and industrial applications. For electronics and electrical components, the system may monitor temperature, impedance, and signs of electrical failure in electronic devices and circuits to prevent overheating and detect early signs of component failure.

These diverse applications demonstrate the versatility of the thermo-electrical wireless sensor system in providing real-time, long-term monitoring across various industries and materials.

The thermo-electrical wireless sensor system may comprise a multi-tiered architecture designed for efficient data collection, transmission, processing, and visualization. At the foundation of this architecture are the sensor nodes, which may form the primary data collection points within the system. Each sensor node may be equipped with thermal sensors and electrical impedance sensors, enabling the precise measurement of temperature and electrical properties of the surrounding environment or material. These sensors may be coupled with a wireless communication transceiver, facilitating the transmission of collected data to other components within the system.

To ensure long-term, autonomous operation, each sensor node may incorporate a solar panel and battery combination. This power system may allow the nodes to harvest and store solar energy, reducing or eliminating the need for manual battery replacements or external power sources. A microcontroller within each node may manage data collection and perform initial processing, potentially reducing the volume of raw data that needs to be transmitted and conserving power.

The next tier in the system architecture may consist of one or more gateways. These gateways may serve as intermediaries between the sensor nodes and the cloud infrastructure. Each gateway may be equipped with wireless communication modules capable of receiving data from multiple sensor nodes. The gateways may also possess additional processing power, enabling them to perform preliminary analysis on the incoming data. This local processing may help to filter, aggregate, or compress data before transmission to the cloud, potentially reducing bandwidth requirements and improving overall system efficiency.

At the core of the system architecture may be a cloud or server infrastructure. This centralized platform may be responsible for data storage and advanced processing. The cloud infrastructure may employ sophisticated machine learning algorithms to perform deep analysis on the collected data, identifying long-term trends, anomalies, or patterns that may not be apparent in short-term or localized data sets. This advanced analysis may enable predictive maintenance, early warning systems, or other proactive measures based on the insights derived from the data.

The final component of the system architecture may be the user interface, which may serve as the primary point of interaction for system operators or stakeholders. This interface may take the form of a web or mobile application, providing real-time monitoring capabilities and data visualization tools. An integral part of this interface may be an alert system, designed to notify users of any anomalies detected by the system or to provide predictions about future maintenance needs based on the analysis performed in the cloud. This user-centric design may enable quick decision-making and responsive management of the monitored environment or infrastructure.

The thermo-electrical wireless sensor system may incorporate advanced machine learning capabilities to enhance its functionality and performance. Machine learning algorithms may analyze the data collected by the sensors to identify patterns, predict future conditions, and detect anomalies. This data analysis capability may enable predictive maintenance, allowing the system to forecast potential failures or maintenance needs, thereby improving reliability and reducing downtime. The machine learning models employed may be adaptive, capable of adjusting to changing conditions and improving their accuracy over time as more data becomes available.

In terms of performance prediction, machine learning may analyze data to accurately forecast the performance of materials. When integrated with real-time monitoring sensors, these predictions may enable immediate adjustments to optimize material designs. The system may also feature an autocalibration capability powered by machine learning. This may allow the sensor system to self-calibrate by continuously learning from real-time data. Such an adaptive process may maintain high accuracy and compensate for changes without manual intervention. Furthermore, the system may be able to predict and address potential failures, thereby reducing downtime and enhancing overall reliability.

The advantages of this system include remote monitoring, enables monitoring of geographically dispersed locations with minimal infrastructure requirements. Autonomous operation, solar recharge capability extends operational longevity, reducing maintenance costs and environmental impact. Scalability, easily scalable by adding additional sensor nodes to expand monitoring coverage or enhance data resolution. Versatility, applicable across various sectors including agriculture, environmental monitoring, infrastructure management, and industrial automation.

The invention of a thermo-electrical wireless sensor system utilizing high-frequency electrical impedance measurement and solar recharge capability represents a significant advancement in remote monitoring technology. Its integration of robust communication, precise sensing capabilities, and sustainable power solutions enables efficient management of resources and infrastructure across diverse applications. The system's potential to enhance operational efficiency, reduce environmental impact, and support data-driven decision-making underscores its relevance in modern sensor network deployments.

Machine learning analyzes data from the concrete mixture to predict its compressive strength accurately. Integrated with real-time monitoring sensors, these predictions enable immediate adjustments to optimize the mixture. This ensures the concrete's structural integrity and compliance with safety standards.

Machine learning analyzes data from the concrete mixture to predict its compressive strength accurately. Integrated with real-time monitoring sensors, these predictions enable immediate adjustments to optimize the mixture. This ensures the concrete's structural integrity and compliance with safety standards. The system utilizes advanced algorithms to process data collected from the thermo-electrical sensors, including temperature, electrical impedance, and other relevant parameters. By analyzing historical data and current measurements, the machine learning model can forecast the concrete's compressive strength at various curing stages, such as 7-day, 28-day, and 56-day intervals.

This predictive capability allows for proactive management of concrete quality throughout the construction process. For example, if the system predicts that a particular batch of concrete may not meet the required strength specifications, it can alert project managers to take corrective actions. These actions might include adjusting the water-to-cement ratio, modifying curing conditions, or even recommending additional reinforcement measures.

The real-time nature of this analysis enables rapid decision-making on construction sites. Instead of waiting for traditional laboratory tests, which can take days or weeks, project teams can make informed choices about formwork removal, load application, or further construction phases based on the AI-driven predictions. This not only enhances safety but also potentially accelerates project timelines and reduces costs associated with delays or concrete failures.

Furthermore, the machine learning system can adapt to various environmental conditions and concrete mix designs. It continuously learns from new data, improving its predictive accuracy over time. This adaptability makes the system valuable across different geographic locations and construction projects, as it can account for local variations in materials, climate, and construction practices.

Machine learning allows sensors to self-calibrate by continuously learning from real-time data. This adaptive process maintains high accuracy and compensates for environmental changes without manual intervention. Additionally, the system can predict and address potential failures, reducing downtime and enhancing reliability.

The machine learning capabilities of the thermo-electrical wireless sensor system enable sophisticated self-calibration and predictive maintenance features. As the sensors continuously collect real-time data on temperature, electrical impedance, and other relevant parameters, the machine learning algorithms analyze this information to refine and adjust the sensor calibration. This adaptive process allows the system to maintain high accuracy even as environmental conditions change over time, such as shifts in ambient temperature, humidity, or the chemical composition of the monitored material.

The self-calibration feature is particularly valuable in long-term monitoring applications, where manual recalibration would be impractical or costly. For instance, in concrete monitoring, the system can adjust its measurements to account for the changing electrical properties of the concrete as it cures and ages. This ensures that the data remains reliable and comparable throughout the entire monitoring period, from initial pouring to long-term structural health assessment.

Furthermore, the machine learning algorithms can identify patterns and anomalies in the collected data that may indicate potential sensor failures or degradation. By detecting these issues early, the system can alert operators to perform preventive maintenance or replace sensors before they fail completely. This predictive approach to maintenance significantly reduces system downtime and enhances overall reliability.

The ability to predict and address potential failures extends beyond the sensors themselves to the monitored structures or materials. For example, in infrastructure monitoring, the system can analyze trends in concrete resistivity or temperature patterns to forecast the onset of structural issues such as cracking or corrosion. This early warning capability allows for timely interventions, potentially preventing costly repairs or catastrophic failures.

By combining self-calibration, adaptive learning, and predictive maintenance, the machine learning-enhanced sensor system provides a robust and reliable solution for long-term monitoring across various applications, from construction and civil engineering to environmental monitoring and industrial process control.

The features, structures, or characteristics described above may be combined in one or more embodiments in any suitable manner, and the features discussed in the various embodiments are interchangeable, if possible. In the following description, numerous specific details are provided in order to fully understand the embodiments of the present disclosure. However, a person skilled in the art will appreciate that the technical solution of the present disclosure may be practiced without one or more of the specific details, or other methods, components, materials, and the like may be employed. In other instances, well-known structures, materials, or operations are not shown or described in detail to avoid obscuring aspects of the present disclosure.

Although the relative terms such as “on,” “below,” “upper,” and “lower” are used in the specification to describe the relative relationship of one component to another component, these terms are used in this specification for convenience only, for example, as a direction in an example shown in the drawings. It should be understood that if the device is turned upside down, the “upper” component described above will become a “lower” component. When a structure is “on” another structure, it is possible that the structure is integrally formed on another structure, or that the structure is “directly” disposed on another structure, or that the structure is “indirectly” disposed on the other structure through other structures.

In this specification, the terms such as “a,” “an,” “the,” and “said” are used to indicate the presence of one or more elements and components. The terms “comprise,” “include,” “have,” “contain,” and their variants are used to be open ended, and are meant to include additional elements, components, etc., in addition to the listed elements, components, etc. unless otherwise specified in the appended claims. The terms “first,” “second,” etc. are used only as labels, rather than a limitation for a number of the objects.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. A sensor system, comprising:
a sensor node comprising: a sensor enclosure; a solar panel positioned relative to a surface of the sensor enclosure; at least one probe connection port; and
processing circuitry housed within the sensor enclosure; and
a sensor probe detachably attachable to the sensor node, the sensor probe comprising: a probe enclosure; a thermocouple positioned within the probe enclosure for temperature measurement; at least two conductive contact points (contacts) positioned within the probe enclosure for electrical measurements; and a cable connecting the probe enclosure to the sensor node via the at least one probe connection port;
wherein the processing circuitry is configured to:
receive temperature data from the thermocouple;
receive electrical measurement data from the at least two conductive contact points;
process the received temperature data and electrical measurement data; and
wirelessly transmit the processed data to an external device.

2. The sensor system of claim 1, wherein the processing circuitry is further configured to:
perform high-frequency electrical impedance measurements using the at least two conductive contact points; and
calculate electrical resistivity of a surrounding medium based on the high-frequency electrical impedance measurements.

3. The sensor system of claim 1, wherein the solar panel is configured to provide power to the sensor node for autonomous operation.

4. The sensor system of claim 1, wherein the sensor node further comprises a rechargeable battery charged by the solar panel.

5. The sensor system of claim 1, wherein the processing circuitry is further configured to implement machine learning algorithms to analyze the processed data and predict future conditions of a monitored medium.

6. The sensor system of claim 1, wherein the at least two conductive contact at formed of at least one of: copper, steel, iron, and gold.

7. The sensor system of claim 1, wherein the sensor node comprises multiple probe connection ports for connecting multiple sensor probes.

8. The sensor system of claim 1, wherein the processing circuitry is further configured to:
determine at least one of water-to-cement ratio, compressive strength, setting time, or transport properties of concrete based on the processed data.

9. The sensor system of claim 1, wherein the sensor probe is configured to be embedded in a material selected from the group consisting of concrete, soil, wood, polymers, composites, thermoplastics, metals, rocks, ceramics, liquid materials, and any combination thereof.

10. The sensor system of claim 1, further comprising a gateway device comprising a gateway device solar panel, a rechargeable battery electrically coupled to the gateway device solar panel, and processing circuitry configured to:
receive the wirelessly transmitted processed data from the sensor node; and
relay the processed data to a remote server for further analysis and visualization.

11. A method for monitoring a material using a sensor system, the method comprising:
providing a sensor node comprising a sensor enclosure, a solar panel positioned on a surface of the sensor enclosure, at least one probe connection port, and processing circuitry housed within the sensor enclosure;
providing a sensor probe detachably attachable to the sensor node, the sensor probe comprising a probe enclosure, a thermocouple positioned within the probe enclosure for temperature measurement, at least two conductive contact points positioned within the probe enclosure for electrical measurements, and a cable connecting the probe enclosure to the sensor node via the at least one probe connection port;
receiving, by the processing circuitry, temperature data from the thermocouple;
receiving, by the processing circuitry, electrical measurement data from the at least two conductive contact points;
processing, by the processing circuitry, the received temperature data and electrical measurement data; and
wirelessly transmitting, by the processing circuitry, the processed data to an external device.

12. The method of claim 11, further comprising:
performing, by the processing circuitry, high-frequency electrical impedance measurements using the at least two conductive contact points; and
calculating, by the processing circuitry, electrical resistivity of a surrounding medium based on the high-frequency electrical impedance measurements.

13. The method of claim 11, further comprising: powering the sensor node using the solar panel for autonomous operation.

14. The method of claim 11, further comprising: charging a rechargeable battery of the sensor node using the solar panel.

15. The method of claim 11, further comprising: implementing, by the processing circuitry, machine learning algorithms to analyze the processed data and predict future conditions of a monitored medium.

16. The method of claim 11, further comprising: implementing, by the processing circuitry, machine learning algorithms to analyze the processed data and develop automated calibration for material mediums.

17. The method of claim 11, wherein the at least two conductive contact points are coated with a non-reactive materials.

18. The method of claim 11, further comprising: connecting multiple sensor probes to the sensor node via multiple probe connection ports.

19. The method of claim 11, further comprising: determining, by the processing circuitry, at least one of water-to-cement ratio, compressive strength, setting time, or transport properties or other material properties of materials based on the processed data.

20. The method of claim 11, further comprising: embedding the sensor probe in a material selected from the group consisting of concrete, soil, wood, polymers, composites, thermoplastics, metals, rocks, ceramics, liquid materials, and any combination thereof.

21. A sensor system comprising:
a sensor node comprising: a sensor enclosure; a solar panel positioned on a surface of the sensor enclosure; at least one probe connection port; and
processing circuitry housed within the sensor enclosure; and
a sensor probe detachably attachable to the sensor node, the sensor probe comprising: a probe enclosure; a thermocouple positioned within the probe enclosure for temperature measurement; at least two conductive contact points positioned within the probe enclosure for electrical measurements; and a cable connecting the probe enclosure to the sensor node via the at least one probe connection port;
wherein the processing circuitry is configured to:
receive temperature data from the thermocouple;
receive electrical measurement data from the at least two conductive contact points;
process the received temperature data and electrical measurement data using a machine learning model;
generate predictions related to a monitored medium based on the processed data; and
wirelessly transmit the processed data and predictions to an external device.
